(19) United States
(12) Patent Application Publication
(10) Pub. No.: US 2018/0110196 A1
(43) Pub. Date: Apr. 26, 2018

(54) ACCELERATED PRODUCTION OF EMBRYOGENIC CALLUS, SOMATIC EMBRYOS, AND RELATED TRANSFORMATION METHODS

(71) Applicant: Dow AgroSciences LLC, Indianapolis, IN (US)

(72) Inventors: Sivarama Reddy Chennareddy, West Lafayette, IN (US); Dayakar Pareddy, Carmel, IN (US); Tobias M. (Toby) Cicak, Whitestown, IN (US); Rodrigo Sarria, Zionsville, IN (US); Katherine K. Effinger, Lafayette, IN (US); Tejinder Kumar Mall, West Lafayette, IN (US)

(21) Appl. No.: 15/786,916

(22) Filed: Oct. 18, 2017

Related U.S. Application Data
(60) Provisional application No. 62/410,526, filed on Oct. 20, 2016.

(51) Int. Cl. A01H 4/00; A01H 5/10; C12N 15/82; C12N 5/00; C07K 14/425

(52) U.S. Cl. CPC A01H 4/005; A01H 4/008; A01H 5/10; C07K 14/425; C12N 15/8234; C12N 5/0025; C12N 15/8205

(57) ABSTRACT

The invention provides a method for the production of soybean embryogenic callus and somatic embryos. The invention further provides methods of transforming explants that includes generating somatic embryo tissue. The transgenic somatic embryos produced can be used for regenerating stable transgenic plants.

### CROSS-REFERENCE T0 RELATED APPLICATIONS

This application claims the benefit of U.S. Provisional application No. 62/410,526, filed Jan. 12, 2007, entitled “ACCELERATED PRODUCTION OF EMBRYOGENIC CALLUS, SOMATIC EMBRYOS, AND RELATED TRANSFORMATION METHODS”, the disclosure of which is being incorporated by reference in its entirety.

### FIELD OF THE DISCLOSURE

The disclosure relates generally to the production of plant tissue cultures. In particular, the disclosed invention relates to a faster production of embryogenic callus and somatic embryos. Disclosed methods are useful for transformation of soybean tissue and commercial development of transgenic soybeans and soybean products.

### BACKGROUND

Soybean (Glycine max) is one of the most important agricultural crops, with an annual crop yield of more than 200 million metric tons, and an estimated value exceeding 40 billion dollars worldwide. Soybean accounts for over 97% of all oilseed production globally. Thus, reliable and efficient methods for improving the quality and yield of this valuable crop are of significant interest.

Traditional breeding methods for improving soybean have been constrained because the majority of soybean cultivars are derived from only a few parental lines, leading to a narrow germplasm base for breeding. Christou et al., TIBTECH 8:145-151 (1990). Modern research efforts have focused on plant genetic engineering techniques to improve soybean production. Transgenic methods are designed to introduce desired genes into the heritable germline of crop plants to generate elite plant lines. The approach has successfully increased the resistance of several other crop plants to disease, insects, and herbicides, while improving nutritional value.

Two general methods are commonly used for transferring genes into soybean plant tissue: Agrobacterium-mediated gene transformation and biolistic transformation (such as by high velocity microparticle bombardment). However, soybeans can be a challenging system for transgenic engineering. Efficient transformation and regeneration of soybean explants is difficult to achieve, and frequently hard to repeat.

Agrobacterium tumefaciens, a pathogenic, soil-dwelling bacterium, has the ability to transfer its DNA, called T-DNA, into host plant cells and to induce the host cells to produce metabolites useful for bacterial nutrition. Using recombinant techniques, some or all of the T-DNA may be replaced with a gene or genes of interest, creating a bacterial vector useful for transforming the host plant. Agrobacterium-mediated gene transfer is typically directed at undifferentiated cells in tissue culture, but may also be directed at differentiated cells taken from the leaf or stem of the plant. In comparison to other methods, Agrobacterium-mediated transformation of soybean can provide certain advantages, including lower number of transgene copy integration, low operating costs and relatively simple protocols. However, Agrobacterium transformation can present challenges, including lower transformation rate and lower transformation efficiency. Furthermore, different soybean cultivars demonstrate wide differences in their susceptibility to Agrobacterium-mediated transformation. Some cultivars are considered recalcitrant to Agrobacterium-mediated transformation. See e.g., Wiebke-Strohm et al. (2012) in Genetic Engineering—Basics, New Applications and Responsibilities, Barrera-Seldana (Ed.) (InTech), pp. 145-172, at 159-160.

Biolistic transformation involves the use of particles to puncture plant cell walls and membranes and to deliver nucleic acids inside the cells. Methods of biolistic transformation include high velocity particle bombardment of specialized tissues such as apical meristem or embryogenic tissue cultures. While methods for biolistic transformation of apical meristem can be completed in 7-10 months (Rech et al. (2008) Nature Protocols 3(3): 410-418), organogenesis from the bombarded meristem is of multicellular origin and, therefore, can produce chimeric plants having transgenic and non-transgenic sectors (Cho et al. (1997) Plant Biotechnol. 14(1): 11-16). By contrast, biolistic transformation methods that include somatic embryogenic callus tissue culture and/or embryogenic suspension cultures are more likely to produce non-chimeric regenerated plants. However, methods that use embryogenic callus cultures can be quite time consuming and complex. For example, the Ohio State Plant Transformation Laboratory has reported that particle bombardment of using its “D20 transformation” of embryogenic tissue typically takes nine to twelve months to recover a transgenic soybean plant. The same laboratory has indicated that the use of embryogenic suspension cultures is technically more demanding and more challenging than the “D20 transformation” method. Additionally, transformation of embryogenic callus-derived materials can result in low transformation frequency and sterility problems (Finer et al. (1991) In Vitro Cell Dev. Biol., 27P: 175-182, and Cho et al. (1997) at 15-16).

Accordingly, there is a desire to improve methods of culturing embryogenic callus and its use in the transformation of soybean.

### SUMMARY OF THE DISCLOSURE

The invention is based, in part, on the discovery that abiotic stress treatment of immature soybean embryo explants can improve their ability to form embryogenic callus. In certain embodiments, the disclosed invention can be used to improve embryogenic callus formation, somatic embryo formation, and somatic embryo regeneration. For example, disclosed methods successfully induced somatic embryogenesis in 60%, 70% or in some cases more than 80% of starting immature embryo explants. Additionally, immature explants that were abiotically stressed in accordance with the invention were regenerated into rooted plants in 14-18 weeks.

The invention is also based, at least in part, on the discovery that cold treatment of soybean embryos can be used to improve gene transfer into soybean tissue, which then can be used to regenerate transgenic soybean tissue and/or transgenic soybean plants. In certain embodiments of the invention disclosed herein, treatment of plant embryo tissue with abiotic stress advantageously provides a faster, less time-consuming method for generating transgenic somatic embryo tissue. The invention also provides improved methods for the transformation of soybean varieties that are considered “recalcitrant” to other methods of transformation.

In one aspect, the invention provides a method of subjecting immature soybean embryo explants to cold treatment and generating embryogenic callus. The immature embryo explant can be a whole zygotic immature embryo or it can be a part thereof, e.g., a split-seed explant, hypocotyl, epicotyl, embryonic axis, cotyledon, or any other portion of the immature embryo capable of generating embryogenic callus. In one embodiment of this aspect, the invention can further include inducing the formation of somatic embryo tissue subsequent to the cold-treatment. Somatic embryo tissue formed using the methods of the invention can be cultured and/or propagated, for example, on semi-solid media or in liquid cultures.

In another aspect, the invention provides a method of transformation that includes subjecting immature soybean embryo explant to cold treatment, generating somatic embryo tissue, and transforming the somatic embryo tissue with one or more exogenous genes (“transgenes”) to generate transgenic somatic embryo tissue. Transformation of somatic embryo tissue can be by any suitable method, for example, by Agrobacterium- or biolistics-mediated transformation. In certain embodiments, this aspect of the invention can further include regenerating transgenic somatic embryo tissue into a stable transgenic plant containing the one or more transgenes. In other embodiments, this aspect of the invention can further include culturing transformed somatic embryo tissue without regenerating a stable transgenic plant, e.g., for transient transformation.

In yet another aspect, the invention provides a method that includes subjecting an immature soybean embryo explant to cold treatment, transforming the embryo explant with one or more transgenes, and subsequently generating transgenic somatic embryo tissue from the transformed embryo explant. Transformation can be done by any suitable method, for example, by Agrobacterium- or biolistics-mediated transformation. In certain embodiments, this aspect of the invention can further include regenerating the transgenic somatic embryo tissue into a stable transgenic plant containing the one or more transgenes. In other embodiments, this aspect of the invention can further include culturing transformed somatic embryo tissue without regenerating a stable transgenic plant, e.g., for transient transformation.

In a further aspect, the invention provides a method of transformation that includes subjecting immature soybean embryo explant to cold treatment and to plasmolysis. In one embodiment of this aspect, the invention includes subjecting immature soybean embryo explant to cold treatment and subsequently subjecting cold-treated explant to plasmolysis to generate twice abiotically-stressed embryo tissue. In another embodiment, the invention includes subjecting immature soybean embryo explant to plasmolysis and subsequently subjecting it to cold-treatment, to thereby generate twice abiotically-stressed embryo explant. In either of these embodiments, the invention can further include using the twice abiotically-stressed embryo explant to generate somatic embryo tissue. Optionally, somatic embryo tissue is maintained or propagated in culture. The somatic embryo tissue can be transformed with one or more transgenes to generate transgenic somatic embryo tissue. Transformation of the somatic embryo tissue can be done by any suitable method, for example, by Agrobacterium- or biolistics-mediated transformation. In certain embodiments, this aspect of the invention can further include regenerating transgenic somatic embryo tissue into a stable transgenic plant containing the one or more transgenes. In other embodiments, this aspect of the invention can further include culturing transformed somatic embryo tissue without regenerating a stable transgenic plant, e.g., for transient transformation.

In a different further aspect, the invention provides a method of transformation that includes subjecting immature soybean embryo explant to cold treatment and plasmolysis to generate twice abiotically-stressed embryo explant. This twice abiotically-stressed immature embryo explant is transformed with one or more transgenes to create twice abiotically-stressed transgenic embryo explant. Transformation can be done by any suitable method, for example, by Agrobacterium- or biolistics-mediated transformation. In certain embodiments of this aspect, the invention can further include subsequently using the twice abiotically-stressed, transgenic explant to develop transgenic somatic embryo tissue. In additional embodiments of this aspect, the invention further includes regenerating transgenic somatic embryo tissue into a stable transgenic plant containing the one or more transgenes. In other additional embodiments of this aspect, the invention further includes culturing transgenic somatic embryo tissue without regenerating a stable transgenic plant, e.g., for transient transformation.

In certain preferred embodiments of the invention, an immature embryo explant is subjected to cold treatment, the explant is dissected to form a split seed explant, and the split seed explant is induced to form embryogenic callus. Further, the explant can be further used to form somatic embryo tissue. This somatic embryo tissue can be transformed to generate transgenic somatic embryo tissue. Transformation can be by any suitable method (e.g., Agrobacterium-mediated or biolistic-mediated). The transgenic somatic embryo can be maintained or propagated on semi-solid media or in liquid cultures. Additionally, the transgenic somatic embryo can be regenerated into stable transgenic plant containing the one or more transgenes.

In other preferred embodiments, the immature embryo explant is subjected to cold treatment, then it is dissected to form a split seed explant, and the split seed explant is subjected to plasmolysis to thereby generate twice abiotically-stressed immature embryo explant. This twice abiotically-stressed immature embryo explant is transformed with one or more transgenes to create twice abiotically-stressed, transgenic immature embryo explant. Transformation can be by any suitable method (e.g., Agrobacterium-mediated or biolistic-mediated). The transgenic immature embryo explant can then be used to generate transgenic somatic embryo. Transgenic somatic embryos can be maintained or propagated in culture or regenerated into a stable transgenic plant containing the one or more transgenes.

In yet other preferred embodiments, the immature embryo explant is subjected to cold treatment, then it is dissected to form a split seed explant, and the split seed explant is subjected to plasmolysis to thereby generate twice abiotically-stressed immature embryo explant. This twice abiotically-stressed immature embryo explant is induced to form embryogenic callus. Further, the explant can be further used to form somatic embryo tissue. The transgenic somatic embryo can be maintained or propagated on semi-solid media or in liquid cultures. In specific examples of this embodiment, the somatic embryo tissue thus generated is transformed with one or more transgenes to create transgenic somatic embryo tissue. Transformation can be by any suitable method (e.g., Agrobacterium-mediated or biolistic-mediated). The transgenic somatic embryo can be maintained or propagated in culture or regenerated into a stable transgenic plant containing the one or more transgenes.

In another aspect, the invention provides a rapid method for biolistic-mediated transformation of immature embryos. In this aspect, the invention includes subjecting immature soybean embryo explant to cold treatment, using biolistic-mediated transformation to transform the embryo explant with one or more transgenes that includes a selectable marker gene, and subsequently generating transgenic somatic embryo tissue from the transformed embryo explant. In a preferred embodiment, the transformed immature soybean embryo explant includes an intact embryonic axis. The embryonic access can be the target for biolistic transformation, e.g., the target for particle bombardment. In some embodiments of this aspect of the invention, the selectable marker gene provides resistance against a selection agent comprising glufosinate. For example the selectable marker gene can be a PAT (phosphinothricin-N-acetyltransferase) gene, a BAR (bialaphos resistance) gene or a DSM2 (Dow selectable marker) gene. In certain embodiments of this aspect of the invention, the selectable marker gene provides resistance against a selection agent comprising glyphosate or an antibiotic such as hygromycin. For example, the selectable marker gene can be a DGT28 (Dow glyphosate tolerance) gene or a HPT (hygromycin phosphotransferase) gene. In further embodiments of this aspect of the invention, the method further includes regenerating the transgenic somatic embryo tissue into a stable transgenic plant containing the one or more transgenes. In some examples of such further embodiments, the stable transgenic plant can be advantageously regenerated from somatic embryo tissue within a period of four to five months after the biolistic-mediated transformation.

### DETAILED DESCRIPTION

In describing and claiming the invention, the following terminology will be used in accordance with the definitions set forth below. Unless otherwise indicated, the terms “a” and “an” as used herein refer to at least one.

As used herein, a “cotyledon” may generally refer to an embryonic leaf or “primary leaf” of the embryo of a seed plant. A cotyledon is also referred to in the art as a “seed leaf.” Dicotyledonous species, such as soybean, have two cotyledons. The “cotyledonary node” refers to the point of attachment of the cotyledons to the embryo in the seed or seedling, and may generally refer to the tissue associated with that point of attachment.

The terms “embryonic axis” or “embryo axis” refer to the major portion of the embryo of the plant, and generally includes the epicotyl and hypocotyl.

The term “explant” refers to a piece of soybean tissue that is removed or isolated from a donor plant (e.g., from a donor seed or immature embryo), cultured in vitro, and is capable of growth in a suitable media.

The “epicotyl” is that portion of the plant embryo or seedling above the cotyledons and below the first true leaves. In the seed, the epicotyl is found just above the cotyledonary node, and may variously be referred to as the “embryonic shoot” or “future shoot.”

The “hypocotyl” is that portion of the plant embryo or seedling below the cotyledons and above the root or radicle (embryonic root). In the seed, the hypocotyl is found just below the cotyledonary node, and may also be referred to as the “hypocotyledonous stem” or the “embryonic stem.” As used herein, the hypocotyl may refer to the location, as the tissue found therein. As used herein, the epicotyl may refer to the location, as described, or the tissue found therein.

The term “plant” refers to either a whole plant, plant tissue, plant part, including pollen, seeds, or an embryo, plant germplasm, plant cell, or group of plants.

The term “plant parts” refers to any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant. Plant cells, as used herein, includes protoplasts and protoplasts with a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant part” in embodiments herein.

The term “dicot” or “dicotyledonous” refers to plants having two cotyledons. Examples include crop plants such as soybean, sunflower, cotton, canola, rape, and mustard.

The term “monocot” or “monocotyledonous” refers to plants having a single cotyledon. Examples include crop plants such as maize, rice, wheat, oat, and barley.

As used herein the term “transformation” refers to the transfer and integration of a nucleic acid or fragment into a host organism, resulting in genetically stable inheritance. Host organisms containing the transformed nucleic acid fragments are referred to as “transgenic” or “recombinant” or “transformed” organisms. Known methods of transformation include Agrobacterium tumefaciens- or Agrobacterium rhizogenes-mediated transformation, calcium phosphate transformation, polybrene transformation, protoplast fusion, electroporation, ultrasonic methods (e.g., sonoporation), liposome transformation, microinjection, naked DNA, plasmid vectors, viral vectors, biolistics (microparticle bombardment), silicon carbide WHISKERS™ mediated transformation, aerosol beaming, or PEG transformation as well as other possible methods.

As used herein, the term “transgenic” refers to a plant cell, plant tissue, plant part, plant germplasm, or plant which is genetically modified, e.g., to include a preselected nucleic acid sequence that has been introduced into the genome of a plant cell, plant tissue, plant part, plant germplasm, or plant by transformation or gene editing technology.

As used herein, the term “transgenic,” “heterologous,” “introduced,” or “foreign” nucleic acid (e.g., DNA, RNA, or gene) refer to a recombinant nucleic acid sequence or gene that does not naturally occur in the genome of the plant; rather the recombinant nucleic acid sequence is artificially incorporated into the organism's genome as a result of human intervention. Such recombinant nucleic acid sequence can be one that is artificially created, one that is from a different species, or one that is found at a different location or association in the genome of the untransformed, non-transgenic plant.

Soybean Type. The invention provides a method for initiating the formation of embryogenic callus in different soybean varieties. In some embodiments, the disclosed methods of the invention are used to initiate embryogenic callus from a soybean that is responsive to embryogenesis induction. Examples of soybean varieties that have been identified as responsive to somatic embryogenesis include, but are not limited to Jack, Kunitz, Council, Cisne, Savoy, and Maverick. In certain embodiments, the invention can be used to initiate embryogenic callus from a soybean that is poorly or moderately responsive to embryogenesis induction. Examples of soybean varieties that have been identified as having poor or moderate response to somatic embryogenesis include, but are not limited to Olympus, Delsoy 500, NE3399, Benning, and MN301. Ko et al. (2004) Crop Sci. 44:1825-1831. In other embodiments, the invention can be used to initiate embryogenic callus from a soybean that has been identified “recalcitrant” to somatic embryogenesis, i.e., very poor or non-embryogenic. Examples of soybean varieties that are recalcitrant to somatic embryogenesis include, but are not limited to Stonewall, Pennyrile, Defiance, Glacier, Cook, MN1301, KS4895, Haskell, and NC Roy. See e.g., Meurer et al. (2001) In Vitro Cell. Dev. Biol.—Plant 37:62-67.

Additionally, the disclosed methods of transformation can be used to transform soybean varieties that are recalcitrant (have poor responsiveness) to efforts of regenerating transformed plants from transgenic callus. Some soybean varieties, like Jack, produce somatic embryogenic callus under a variety of conditions, and callus tissue in these varieties can be proliferated by repeated subculturing. By contrast, other soybean varieties, those referred to as recalcitrant herein, are poorly embryogenic or non-embryogenic using established methods. See e.g., Meurer et al. (2001). Therefore, the methods of the present invention can be used to improve the responsiveness of recalcitrant soybean varieties using abiotic stress-treatment, transformation, and regeneration processes disclosed herein.

Pod Selection and Harvesting. Any pod containing suitable immature embryos can be used in the methods of the invention. In some embodiments, the methods of the invention use immature embryo which has been harvested from a soybean selected based on age, size, number of embryos, or a combination of these characteristics. For example, pods containing embryos for use in the invention can be harvested from plants having an age of four to six weeks, e.g., five weeks after planting. In certain embodiments, pods containing embryos for use in the invention are harvested from plants about seven to fourteen days after flowering. Additionally, or alternatively, the pods having a width larger than 5 mm, larger than 6 mm, larger than 7 mm, or larger than 8 mm are selected that contain immature embryos for use in the methods of the invention. In particular embodiments, pods larger than 9 mm in width are selected that contain immature embryos for use in the methods of the invention. In further embodiments, pods containing a suitable number of immature embryos are selected using any suitable method of embryo detection. For example, a trans-illuminated stereoscope can be used to identify pods having two or three immature embryos for use in the invention.

Preferably, the pods containing immature embryos are surface cleaned or surface sterilized (e.g. by washing with ethanol and/or bleach solutions, followed by sterile water rinse) prior to further use in connection with the invention.

Treatment of Embryos with Cold Abiotic Stress. In the methods of the invention, immature embryos are treated with cold abiotic stress. In one embodiment of the invention, pods (preferably surface cleaned or sterilized pods) containing the immature embryos are subjected to cold treatment and subsequently processed for callus initiation and induction. In one example, pods containing embryos are first subjected to cold treatment, subsequently one or more immature embryo explants are separated from the remaining pod tissues, and the one or more embryo explants are placed on suitable media to form callus. The embryo explant can be a whole embryo explant or it can be any part thereof, which is suitable for callus induction and/or somatic embryo formation. In a further example of this embodiment, pods are cold-treated, one or more immature embryo explants are separated from the remaining pod tissues, and each embryo explant is split to form a “split seed” explant that retains at least a portion of the embryonic axis, more preferably most of the embryonic axis, still more preferably substantially all or all of the embryonic axis. The cold-treated immature embryo explant, now in in its split seed form, is placed on suitable media to form callus. The foregoing callus may be subsequently processed to generate somatic embryo tissue, transformed (e.g., by Agrobacterium- or biolistic-mediated transformation), or both in accordance with the invention disclosed herein.

In another embodiment of the invention, one or more immature embryo explants are first isolated from their pods and subsequently the isolated embryo explants are subjected to cold abiotic stress. In certain examples of this embodiment, the one or more immature embryos are whole and generally intact when subjected to cold treatment. In another embodiment, after removal from their pods, the one or more immature embryos are wounded and subjected to cold treatment. In still another embodiment, after removal for their pods, the one or more immature embryos are split to form a split seed explant that, preferably retains a portion of the embryonic axis, more preferably retains most of the embryonic axis, and more preferably retains substantially all or all of the embryonic axis. This split seed embryo explant is subject to cold treatment.

As used herein cold treatment means subjecting an immature embryo explant (while in its pod or isolated from its pod) to temperatures of 15° C. or less for a period of two or more days.

In one embodiment of the invention, cold treatment includes subjecting an immature embryo to temperatures of 8° C. or less for a period of two or more days. In certain embodiments of the invention, an immature embryo is subject to temperatures in the range of 0° to 8° C. e.g., a temperature of about 8° C., about 7° C., about 6° C., about 5° C., about 4° C., about 3° C., about 2° C., about 1° C., or about 0°, for two to twelve days. For example, immature embryos can be kept at temperatures in the range of 4° C. (±3° C.) or 5° C. (±3° C.). Time period of cold treatment can be two days, three days, four days, five days, six days, seven days, eight days, nine days, ten days, eleven days, or twelve days or more.

In certain embodiments, the immature embryo is subjected to cold treatment at a temperature in the range of 4° to 8° C., e.g., a temperature of about 8° C., about 7° C., about 6° C., about 5° C., or about 4° C. Time period of cold treatment can be two days, three days, four days, five days, six days, seven days, eight days, nine days, ten days, eleven days, or twelve days or more.

In particular embodiments, the immature embryo is subjected to cold treatment at a temperature ranging from 4° to 8° C., e.g., a temperature of about 8° C., about 7° C., about 6° C., about 5° C., or about 4° C., and the time period of cold treatment can be seven to nine days or eight to ten days.

In other embodiments, cold treatment includes subjecting an immature embryo to a temperature ranging from 8° C. to 15° C., e.g., a temperature of about 14° C., about 13° C., about 12° C., about 11° C., about 10° C., about 9° C., or about 8° C., for a treatment period of two to twelve days. Thus, immature embryos can be kept at any of the foregoing temperatures in the range of 8° C. to 15° C. for two days, three days, four days, five days, six days, seven days, eight days, nine days, ten days, eleven days, or twelve days or more.

Dissecting Pods, Isolation and Selection of Immature Embryo Explants. Immature embryo explants can be separated from pods using any suitable method. An instrument such as a trans-illuminated microscope can be used to determine the position and approximate size of immature embryos within the pods. Pods containing appropriate number and size of immature embryos can be selected for dissection. For example, a pod can be dissected by initially making two cuts on both ends, then cutting longitudinally along the curved part of the pod, removing and disassembling sufficient pod tissue to expose the interior pod cavity. Seeds containing immature embryos can then be detached from the pod tissue.

In certain embodiments of the invention, immature embryos are selected for use based on their length. In the context of the invention, the “length” of an immature embryo can be determined, for example, by using a caliper or analyzing an image of the isolated embryo and determining the maximum Feret measurement of the object. Generally, a Feret measurement refers to the maximum distance between two parallel tangential lines restricting an object.

In some embodiments of the invention, immature embryos selected for use have a length of 2 mm or more, a length of 3 mm or more, length of 4 mm or more, a length of 5 mm or more, or a length of 6 mm or more. In some embodiments, immature embryos selected for use have a length of 10 mm or less, 9 mm or less, a length of 8 mm or less, a length of 7 mm or less, or a length of 6 mm or less.

In further embodiments of the invention, immature embryos selected for use have a length of 2 mm to 10 mm. For example, immature embryos can be selected having a length of 3 mm to 10 mm, a length of 3 mm to 9 mm, a length of 3 mm to 8 mm, a length of 3 mm to 7 mm, a length of 3 mm to 6 mm, or a length of 3 mm to 5 mm. In other examples, immature embryos can be selected having a length of 4 mm to 10 mm, a length of 4 mm to 9 mm a length of 4 mm to 8 mm, a length of 4 mm to 7 mm, or a length of 4 mm to 6 mm. In still other examples example, immature embryos can be selected having a length of a length of 5 mm to 10 mm, a length of 5 mm to 9 mm a length of 5 mm to 8 mm, or a length of 5 mm to 7 mm. In further examples, immature embryos can be selected having a length of 6 mm to 10 mm, a length of 6 mm to 9 mm, or a length of 6 mm to 8 mm.

Bisecting Embryos to form Split Seeds and Other Preparations of Embryos. The term “split seed” or “split immature embryo” as used herein refers to the practice of splitting an embryo containing seed. In one embodiment of the invention, seeds are separated from the pod and, optionally having selected seeds based on immature embryo length, the soybean seed can be split longitudinally along the hilum of the soybean seed, thereby bisecting the seed. Methods of bisecting seed are disclosed for example in U.S. Patent Application Publication 2014.0173774 (Pareddy et al.) and U.S. Pat. No. 7,473,822 (Paz et al.). Thus, in one example of the embodiment, the soybean seed is split so that the embryonic axis remains attached to the nodal end of the soybean seed. The embryonic axis which is retained with the soybean seed can comprise any portion of the embryonic axis seed structure. As such, any portion or amount of embryonic axis which is retained while splitting soybean the seed is within the scope of the disclosure. Embodiments include any portion of embryonic axis that is retained with the splitting of the soybean seed comprising any full length portion or any partial portion or embryonic axis. For example, ¼, ⅓, ½, ⅔, or ¾ of the embryonic axis may be retained when splitting the soybean seed. In another example, none of the embryonic axis is removed and the split seed retains its full length. In splitting the immature embryo, seeds is preferably prepared by splitting the cotyledons of the seeds along the hilum to separate the cotyledons, then removing the seed coat. The embryonic axis (the full length or retained portion) remains attached to the cotyledons.

Alternatively, seeds can be prepared in other ways. For example, while wounding of soybean seed is not required with the disclosed method, it has been reported to increase transformation efficiency using methods such as the cotyledonary node (“cot node”) method and the meristem explant method for Agrobacterium transformation. See U.S. Pat. Nos. 7,696,408 (Olhoft, et al.) and 6,384,301 (Martinelli et al.). Wounding of the plant material may be facilitated by cutting, abrading, piercing, sonication, plasma wounding, or vacuum infiltration. Further, in certain embodiments of the method, wounding can be combined with the disclosed split seed method.

Embryogenic Callus Initiation. After cold treatment of immature embryos in accordance with the invention, one or more cold-treated immature embryo are placed on media under conditions that initiate embryogenic callus formation. In some embodiments, the cold-treated embryo is prepared such as by wounding or by splitting to form a split seed embryo explant prior to initiation of embryogenic callus.

In the methods of the invention, cold-treated immature embryo explants are induced to form embryogenic callus using any suitable method. For example, after cold treatment, immature embryos can be exposed to one or more auxins such as alpha-naphthaleneacetic acid (“NAA”) or, more commonly, 2,4-dichlorophenoxyacetic acid (“2,4-D”) in growth media (see, e.g., Wiebke-Strohm et al. (2012) at 146-148). Cold treated immature embryo explants can be cultured in the presence of an auxin, e.g., NAA or 2,4-D-containing semi-solid media or liquid media. For example, cold-treated immature embryos can be cultured in semi-solid media containing of 2,4-D at concentrations from 20 mg/L to 40 mg/L. See e.g., Finer et al. (1988) Plant Cell, Tissue and Organ Culture, 15: 125-136, disclosing semi-solid callus induction media comprised of MS salts, 40 mg/ml 2,4-D, Gamborg's B5 vitamins, 6% sucrose, and 15 mM glutamine (pH 5.7). In another example, cold-treated immature embryos can be cultured in liquid suspension media containing of 2,4-D at lower concentrations. See, e.g., Finer et al. (1988) and Finer et al. (1991) In vitro Cell Dev. Biol. 27P: 175-182, disclosing the use of suspension medium 10A40N, including variations and optimized versions thereof, for the initiation of embryogenic callus. Disclosed 10A40N suspension media comprises modified MS salts (MS nitrogen replaced with 10 mM NH₄NO₃ and 30 mM K NO₃), 5 mg/ml 2,4-D, Gamborg's B5 vitamins, 6% sucrose, and 15 mM glutamine (pH 5.7).

In particular embodiments, embryos are selected based on their size before placing on callus initiation media. See e.g., disclosure regarding “Dissecting Pods, Isolation and Selection of Immature Embryo Explants” above and Examples below demonstrating that size selection can improve the frequency of callus formation.

Embryogenic callus generated from cold-treated embryos as disclosed herein, can be used for any suitable applications. In some embodiments of the invention, the callus-bearing immature embryo is transformed with exogenous DNA, e.g., by biolistic- or Agrobacterium-mediated transformation. In other embodiments of the invention, the callus-bearing immature embryo is subjected to plasmolytic treatment and then, optionally, transformed with exogenous DNA, e.g., by biolistic- or Agrobacterium-mediated transformation. Methods for plasmolysis treatment as well as methods for biolistic transformation and Agrobacterium transformation are known and described herein.

In certain embodiments of the invention, the callus-bearing immature embryo is further used to generate somatic embryos. Methods for generating somatic embryos are known and described herein.

Somatic Embryogenesis, Maturation and Proliferation. In some embodiments, the methods of the invention include culturing immature embryos at least until early somatic embryo (“SE”) formation can be observed within several weeks (e.g., three weeks) on callus initiation media. In further embodiments, the methods of the invention include further culturing such embryos under conditions that promote the formation of somatic embryo, i.e., somatic embryogenesis.

In some embodiments of the invention, immature embryos with early somatic embryos are removed from auxin-containing media and immature embryos are transferred to somatic embryo maturation media lacking auxin or 2,4-D. For example, such media can be liquid or semi-solid. Auxin-free maturation media is described, for example, as “MSO” media in Durham et al. (1992) Plant Cell Reports, 11:122-125. See also somatic embryo germination and maturation (SEGM) media and soybean histodifferentiation and maturation (SHaM) media described herein.

In other embodiments of the invention, immature embryos can be maintained on and/or subcultured onto fresh batch of media (e.g., liquid or semi-solid media) containing auxin such as alpha-naphthaleneacetic acid (NAA) or 2,4-D. For example, continued culture and proliferation of embryogenic material in the presence of auxin-containing media at the same or at lower concentrations. For example Meurer et al. (2001) In Vitro Cell Dev. Biol., 37:62-67 discloses that after callus initiation on “SD40” media containing 40 mg/ml 2,4-D, callus induced explants were transferred to “SD20” culture media containing 20 mg/L 2,4-D for the development and proliferation of somatic embryos. Hofmann et al. (2004) Plant, Cell Tissue and Organ Culture, 77: 157-163 discloses callus initiation and somatic embryo formation on media containing a variety of concentrations of either NAA or 2,4-D.

In certain embodiments of the invention, somatic embryos developed in the presence of auxin or 2,4-D are transferred to suspension culture media. In further embodiments, somatic embryo suspension cultures can be maintained or passaged in media such as 10A40N. See e.g., Finer et al. (1988), describing optimized procedures and media for passaging and proliferation of somatic embryo suspension cultures. See also, Santarem et al. (1998) Plant Cell Reports, 17:752-759 describing the use of Agrobacterium to transform somatic embryos from suspension cultures for transient expression.

Somatic embryo cultures developed in accordance with the invention can be used for transformation by Agrobacterium and/or biolistic methods such as particle bombardment. See e.g., Finer et al. (1991), Trick et al. (1997) Plant Tissue Culture and Biotech, 3(1): 9-26; and Santarem et al. (1998).

Plasmolysis Treatment of Embryos. In some of the methods of the invention, immature embryos explant are subjected to plasmolysis treatment, in addition to cold treatment. Plasmolysis involves placing the immature embryo in hypertonic media, which promotes the loss of water and reduced turgor pressure of embryo surface cells. In some circumstances, if this state continues, the protoplasm (plasma membrane) can detach from the cell wall. Accordingly, in some embodiments of the invention, immature embryos are exposed to hypertonic media such as a solution or gel containing one or more tonicity agents. Tonicity agents include salts and sugars. For example, plasmolytic media can include one or more sugars such as sucrose, mannitol, sorbitol, glycol, glycerol, inositol, and combinations thereof solution. Other sugars that can be used in plasmolytic media include, for example, erythritol, threitol, arabitol, xylitol, ribitol, dulcitol, iditol, isomalt, maltitol, lactitol, and combinations thereof. In certain examples, plasmolytic media can include one or more water soluble oligomer or polymers, such as polyethylene oxide (PEO), polyoxyethylene (POE), or polyethylene glycol (PEG), which have a wide range of molecular weights (MWs) (e.g., PEG MW3000-4000 or PEG MW4000-8,000). In still other examples, plasmolytic media can include a mixture of (i) one or more sugars and (ii) a water soluble oligomer such as PEG. Suitable plasmolysis media for use in the transformation of plant cells have been described for example in Wu et al. (1999) Plant Cell Rep. 18:381-386, Koscianska et al. (2001), Walker et al. (2001) Plant Cell Tissue Organ Cult. 64: 55-62, and Paz et al. (2006) Plant Cell Rep 25: 206-213 (using 1/10 MS liquid medium (Murashige and Skoog (1962)) with 1.0M sucrose).

In certain embodiments, the plasmolytic media can contain one or more tonicity agents (e.g., salt(s) or sugar(s)) at a molar concentration range of 0.2M to 2.0M, 0.2M to 1.5M, 0.1M to 1.0M 0.2M to 0.8M, 0.2M to 0.6M, 0.2M to 0.4M, 0.4M to 2.0M, 0.4M to 1.0M, 0.4M to 1.5M, 0.4M to 0.8M, 0.4M to 0.6M, 0.6M to 2.0M, 0.6M to 1.5M, 0.6M to 1.0M, 0.6M to 0.8M. In particular embodiments, the plasmolytic solution contains at least one tonicity agents at a molar concentration of 0.2M, 0.3M, 0.4M, 0.5M, 0.6M, 0.7 M, 0.8M, 0.9M, 1.0M, 1.1M, 1.2M, 1.3M, 1.4M, 1.5M, 1.6, 1.7M 1.8M, 1.9M or 2.0M.

The treatment of immature embryo explants to abiotic stress according to the invention can involve culturing the explant on plasmolytic media for 5 minutes, 10 minutes, 15 minutes, 20 minutes, 30 minutes, or 45 minutes. In other embodiments, treatment of immature embryo explants to abiotic stress according to the invention can involve culturing the explant on plasmolytic media from 1 or 2 hours to 24 hours, e.g., from 2 to 16 hours, from 2 to 12 hours, from 2 to 10 hours, from 2 to 8 hours, from 2 to 6 hours, or from 2 to 4 hours. In some embodiments, the explant can be cultured on plasmolytic media from 4 to 24 hours, from 4 to 16 hours, from 4 to 12 hours, from 4 to 10 hours, from 4 to 8 hours, or from 4 to 6 hours. In particular embodiments, the explant is cultured on plasmolytic media from 1 hour, 2 hours, 3 hours, 4 hours, 5 hours, 6 hours, 7 hours, eight hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or longer to thereby treat explant to abiotic stress.

Methods for Optimizing Abiotic Stress Treatment. The invention provides methods for optimizing the abiotic stress treatment to produce desired result: generate the most somatic embryos, most germline transgenic plants, or highest level of transient expression in a transgenic plant or plant part. In certain embodiments, the method is used to optimize the application of abiotic stress to a particular soybean variety, e.g., a soybean type that is responsive or a soybean type that is recalcitrant to the regeneration of transformed plants from transgenic callus.

In one aspect, the invention provides a method for optimizing the temperature, the time, or both the temperature and time that a soybean embryo is subjected to cold treatment to obtain a desired result. The desired result can be (i) an acceptable (or the highest) number or percentage of somatic embryos generated from an initial number of cold-treated immature embryos, (ii) an acceptable (or the highest) number or percentage of stable, germline transgenic plants generated from an initial number of cold-treated immature embryos, or (iii) an acceptable (or the highest) level of transient expression of a gene of interest found in a transgenic plant or plant part regenerated from a cold-treated immature embryo.

Thus, in one embodiment of the invention, immature soybean embryos (e.g., embryos of one or more pre-selected soybean variety) are subjected to cold treatment for varying amounts of time, i.e., multiple times in a range between a shorter time endpoint and a longer time endpoint, and embryos are evaluated to determine whether and/or how well they produce a desired result. The shorter time endpoint of the range can be as short as less than an hour, an hour or two hours. For example, the shorter endpoint of the time range for optimizing cold treatment can be an hour, two hours, three hours, four hours, five hours, six hours, seven hours, eight hours, nine hours, ten hours, eleven hours, twelve hours, fourteen hours, sixteen hours, eighteen hours, twenty hours, twenty two hours, twenty fours, two days or three days. Each of the foregoing shorter time endpoints can be used in a range for optimizing cold treatment that includes a longer time endpoint that is as long as a week, fourteen days or several weeks. For example, each disclosed shorter time endpoint of the range can be used in a range for optimizing cold treatment that includes one of the following longer time endpoints: less than four days, four days, five days, six days, seven days, eight days, nine days, ten days, eleven days, or twelve days or more.

In another embodiment of the invention, immature soybean embryos (e.g., embryos of one or more pre-selected soybean variety) are subjected to a range of varying cold-treatment temperatures, for example, multiple temperatures within the range of from about 0° C. to about 15° C., and embryos are evaluated to determine whether and/or how well they produce a desired result. The lower endpoint of the range used to optimize temperature can be any two times between about 1° C. and 15° C. For example, the lower endpoint of the range used to optimize temperature can be about 1° C., 2° C., 3° C., 4° C., 5° C., or 6° C. Each of the foregoing lower endpoint temperatures can be used in a range that for optimizing temperature that includes one of the following the higher endpoints: about 7° C., 8° C.; 9° C., 10° C., 11° C., 12° C., 13° C., 14° C., or 15° C.

In still another example of each of the foregoing embodiments, soybean embryos are subjected to both varying times (e.g., including times in any of the foregoing time ranges) and varying temperatures (e.g., any of the foregoing temperature ranges) to determine the appropriate time and temperature that produces a desired result (e.g., (i) the highest or acceptable number or percentage of somatic embryos generated from an initial number of cold-treated immature embryos, (ii) the highest or acceptable number or percentage of stable, germline transgenic plants generated from an initial number of cold-treated immature embryos, or (iii) the highest or acceptable level of transient expression of a gene of interest found in a transgenic plant or plant part regenerated from a cold-treated immature embryo). Such an optimization of both time and temperature can be determined using a combinatorial matrix study.

In another aspect, the invention provides a method for optimizing the plasmolysis treatment to obtain a desired result. In various embodiments, the desired result can be (i) an acceptable (or the highest) number or percentage of somatic embryos generated from an initial number of cold-treated immature embryos, (ii) an acceptable (or the highest) number or percentage of stable, germline transgenic plants generated from an initial number of cold-treated immature embryos, or (iii) an acceptable (or the highest) level of transient expression of a gene of interest found in a transgenic plant or plant part regenerated from a cold-treated immature embryo.

Thus, in one embodiment of the invention, soybean embryos (e.g., embryos of a pre-selected soybean variety) are subjected to plasmolysis for varying amounts of time, i.e., multiple times in a range between a shorter time endpoint and a longer time endpoint, and embryos are evaluated to determine whether and/or how well they produce a desired result. The shorter time endpoint of the range can be as short as, for example, a few minutes, an hour or several hours. The longer time endpoint of the range can be about four hours, five hours, six hours, seven hours, eight hours, nine hours, ten hours, eleven hours, twelve hours, fourteen hours, sixteen hours, eighteen hours, twenty hours, twenty-two hours, twenty-four hours, twenty-six hours, twenty-eight hours, thirty hours, thirty-two hours, thirty-four hours, thirty-six hours or more.

In another embodiment of the invention, soybean embryos (e.g., embryos of a pre-selected soybean variety) are subjected to plasmolysis using different tonicity agents and/or different concentrations of tonicity agents, and embryos are evaluated to determine whether and/or how well they produce a desired result.

In a further aspect, the invention provides a method of optimizing both cold treatment and plasmolysis to determine the best time and temperature that produces a desired result. Thus the method comprises varying one or more cold treatment variables as described herein and also varying one or more plasmolysis variables as described herein to determine a desired result. The desired result can be (i) an acceptable (or the highest) number or percentage of somatic embryos generated from an initial number of cold-treated immature embryos, (ii) an acceptable (or the highest) number or percentage of stable, germline transgenic plants generated from an initial number of cold-treated immature embryos, or (iii) an acceptable (or the highest) level of transient expression of a gene of interest found in a transgenic plant or plant part regenerated from a cold-treated immature embryo. Such an optimization of both cold treatment and plasmolysis treatment can be determined using a combinatorial matrix study.

Methods for Transformation. Methods for the genetic engineering of plants by transformation of plant cells are known in the art. Numerous methods for plant transformation have been developed, including biological and physical transformation protocols for dicotyledonous plants as well as monocotyledonous plants (e.g., Goto-Fumiyuki et al., Nature Biotech 17:282-286 (1999); Miki et al., Methods in Plant Molecular Biology and Biotechnology, Glick, B. R. and Thompson, J. E. Eds., CRC Press, Inc., Boca Raton, pp. 67-88 (1993)). In addition, vectors and in vitro culture methods for plant cell or tissue transformation and regeneration of plants are available, for example, in Gruber et al., Methods in Plant Molecular Biology and Biotechnology, Glick, B. R. and Thompson, J. E. Eds., CRC Press, Inc., Boca Raton (1993), pp. 89-119. For example, the DNA construct may be introduced directly into the genomic DNA of a plant cell using techniques such as electroporation and microinjection of plant cell protoplasts, or the DNA constructs can be introduced directly to plant tissue using biolistic methods, such as DNA particle bombardment (see, e.g., Klein et al. (1987) Nature 327:70-73). Additional methods for plant cell transformation include microinjection via silicon carbide WHISKERS™ mediated DNA uptake (Kaeppler et al. (1990) Plant Cell Reporter 9:415-418). Alternatively, the DNA construct can be introduced into the plant cell via nanoparticle transformation (see, e.g., U.S. patent application Ser. No. 12/245,685, which is incorporated herein by reference in its entirety).

A known biolistic method is microprojectile-mediated transformation, in which DNA is carried on the surface of microprojectiles. In this method, the expression vector is introduced into plant tissues with a biolistic device that accelerates the microprojectiles to speeds sufficient to penetrate plant cell walls and membranes. Sanford et al. (1987) Part. Sci. Technol. 5:27; Sanford, J. C. (1988) Trends Biotech. 6:299, Sanford, J. C. (1990) Physiol. Plant 79:206; Klein et al (1992) Biotechnology 10:268.

The use of microprojectiles or microparticles for the transformation of soybean embryogenic tissue, somatic embryo suspension cultures, have been described in Rech et al. (2008) Nature Protocols 3(3): 410-418; Finer et al. (1991) In vitro Cell Dev. Biol. 27P: 175-182; and Finer et al. (1997) Plant Tissue Culture and Biotechnology 3(1): 9-26, e.g., at 13-18. Direct transformation of isolated soybean explants containing embryonic axes has been described in Christou (1992) Plant J. 2(3): 275-281.

A widely used method for introducing a transgene into plants is based on the natural transformation system of Agrobacterium. Horsch et al (1985) Science 227:1229. A. tumefaciens and A. rhizogenes are plant pathogenic soil bacteria known to be useful to genetically transform plant cells. The Ti and Ri plasmids of A. tumefaciens and A. rhizogenes, respectively, carry genes responsible for genetic transformation of the plant. Kado, C. I. (1991) Crit. Rev. Plant. Sci. 10:1. Descriptions of Agrobacterium vector systems and methods for Agrobacterium-mediated gene transfer are also available, for example, Gruber et al., supra, Miki et al., supra, Moloney et al. (1989) Plant Cell Reports 8:238, and U.S. Pat. Nos. 4,940,838 and 5,464,763. A number of procedures have been developed for Agrobacterium-mediated transformation of soybean that may loosely be classified based on the explant tissue subjected to transformation. U.S. Pat. No. 7,696,408 (Olhoft, et al.) discloses a cotyledonary node (“cot node”) method for transforming monocotyledonous and dicotyledonous plants. U.S. Pat. No. 6,384,301 (Martinelli et al.) discloses Agrobacterium-mediated gene delivery into meristem tissue from soybean embryos. In both the “cot node” and meristem methods explants are preferably wounded prior to infection. U.S. Pat. No. 7,473,822 (Paz et al.) discloses a modified cotyledonary node method called the “half-seed explant” method in which the embryonic axis and shoots are completely removed prior to infection, but no other wounding occurs. Agrobacterium-mediated transformation proceeds, potential transformants are selected, and explants are regenerated on selection medium.

The use of Agrobacterium-mediated gene transfer into soybean somatic embryos in suspension cultures, which are then regenerated into transgenic plants has been described in Finer et al. (1997) at 12, 18-19, and 21-22. The use of Agrobacterium-mediated gene transfer into immature embryo explants, which are then induced to form somatic embryos and subsequently regenerated into transgenic plants has been described in Santarem et al. (1999) In Vitro Cell. Dev. Biol. Plant, 35: 451-455; Ko et al. (2004) In vitro Cell Dev. Biol. 40: 552-558; and Ko et al. (2004) Crop Science 44: 1825-1831. A further method of sonication assisted Agrobacterium transformation (“SAAT”) includes sonicating immature embryo explant during infection by Agrobacterium and thereafter using somatic embryo suspension cultures to develop stable transformants is described by Trick et al. (1998) Plant Cell Reports 17: 482-488. SAAT can also be used for the transient transformation of somatic embryo suspension cultures as described in Santarem et al. (1998) Plant Cell Reports, 17:752-759.

If Agrobacterium is used for transformation, the DNA to be inserted should be cloned into special plasmids, namely either into an intermediate vector or into a binary vector. Intermediate vectors cannot replicate themselves in Agrobacterium. The intermediate vector can be transferred into Agrobacterium tumefaciens by means of a helper plasmid (conjugation). The Japan Tobacco Superbinary system is an example of such a system (reviewed by Komari et al., in Methods in Molecular Biology No. 343: Agrobacterium Protocols (K. Wang, ed.) (2n^{d} Edition) HUMANA PRESS Inc., Totowa, N.J., (2006), Vol. 1, pp.15-41; and Komori et al. (2007) Plant Physiol. 145:1155-1160). Binary vectors can replicate themselves both in E. coli and in Agrobacterium. They comprise a selection marker gene and a linker or polylinker which are framed by the right and left T-DNA border regions. They can be transformed directly into Agrobacterium (Holsters, 1978). The Agrobacterium used as host cell is to comprise a plasmid carrying a vir region. The Ti or Ri plasmid also comprises the vir region necessary for the transfer of the T-DNA. The vir region is necessary for the transfer of the T-DNA into the plant cell. Additional T-DNA may be contained.

The virulence functions of the Agrobacterium tumefaciens host will direct the insertion of a T-strand containing the construct and adjacent marker into the plant cell DNA when the cell is infected by the bacteria using a binary T DNA vector (Bevan (1984) Nuc. Acid Res. 12:8711-8721) or the co-cultivation procedure (Horsch et al. (1985) Science 227:1229-1231). Generally, the Agrobacterium transformation system is used to engineer dicotyledonous plants (Bevan et al. (1982) Ann. Rev. Genet 16:357-384; Rogers et al. (1986) Methods Enzymol. 118:627-641). The Agrobacterium transformation system may also be used to transform, as well as transfer, DNA to monocotyledonous plants and plant cells. See U.S. Pat. No. 5, 591,616; Hernalsteen et al. (1984) EMBO J 3:3039-3041; Hooykass-Van Slogteren et al. (1984) Nature 311:763-764; Grimsley et al. (1987) Nature 325:1677-179; Boulton et al. (1989) Plant Mol. Biol. 12:31-40; and Gould et al. (1991) Plant Physiol. 95:426-434.

Following the introduction of the genetic construct into plant cells, plant cells can be grown and upon emergence of differentiating tissue such as shoots and roots, mature plants can be generated. In some embodiments, a plurality of plants can be generated. Methodologies for regenerating plants are known to those of ordinary skill in the art and can be found, for example, in: Plant Cell and Tissue Culture, 1994, Vasil and Thorpe Eds. Kluwer Academic Publishers and in: Plant Cell Culture Protocols (Methods in Molecular Biology 111, 1999 Hall Eds. Humana Press). The genetically modified plant described herein can be cultured in a fermentation medium or grown in a suitable medium such as soil. In some embodiments, a suitable growth medium for higher plants can include any growth medium for plants, including, but not limited to, soil, sand, any other particulate media that support root growth (e.g., vermiculite, perlite, etc.) or hydroponic culture, as well as suitable light, water and nutritional supplements which optimize the growth of the higher plant.

Alternative techniques for transformation for use with cold-treated embryos in accordance with the invention include calcium phosphate transfection, polybrene transformation, protoplast fusion, protoplast transformation through calcium chloride precipitation, electroporation, ultrasonic methods (e.g., sonoporation), liposome transformation, microinjection, naked DNA, plasmid vectors, viral vectors, biolistics (microparticle bombardment), silicon carbide WHISKERS mediated transformation, aerosol beaming, or PEG as well as other possible methods. Alternatively, gene transfer and transformation methods include, but are not limited to polyethylene glycol (PEG)-mediated or electroporation-mediated uptake of naked DNA (see Paszkowski et al. (1984) EMBO J 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505).

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al. (1983) “Protoplasts Isolation and Culture” in Handbook of Plant Cell Culture, pp. 124-176, Macmillian Publishing Company, New York (1983); and Binding, Regeneration of Plants, Plant Protoplasts, pp. 21-73, CRC Press, Boca Raton (1985). Regeneration can also be obtained from plant callus, explants, organs, pollens, embryos or parts thereof. Such regeneration techniques are described generally in Klee et al. (1987) Ann. Rev. of Plant Phys., 38: 467-486.

Unless otherwise specifically defined, all technical and scientific terms used herein have the same meaning as commonly understood by those of ordinary skill in the art to which this disclosure belongs. Definitions of common terms in molecular biology can be found in, for example, Lewin B., Genes V, Oxford University Press, (1994) (ISBN 0-19-854287-9); Kendrew et al. (eds.), The Encyclopedia of Molecular Biology, Blackwell Science Ltd. (1994) (ISBN 0-632-02182-9); and Meyers R. A. (ed.), Molecular Biology and Biotechnology: A Comprehensive Desk Reference, VCH Publishers, Inc. (1995) (ISBN 1-56081-569-8).

It will be appreciated by one skilled in the art that the use of reporter or marker genes for selection of transformed cells or tissues or plant parts or plants can be included in the transformation vectors or construct. Examples of selectable markers include those that confer resistance to anti-metabolites such as herbicides or antibiotics, for example, dihydrofolate reductase, which confers resistance to methotrexate (Reiss, Plant Physiol. (Life Sci. Adv.) 13:143-149; see also Herrera Estrella et al. (1983), Nature 303:209-213; Meijer et al., Plant Mol. Biol. 16:807-820, 1991); neomycin phosphotransferase, which confers resistance to the aminoglycosides neomycin, kanamycin and paromycin (Herrera-Estrella (1983), EMBO J. 2:987-995; and Fraley et al. (1983) Proc. Natl. Acad. Sci. USA, 80:4803) and hygromycin phosphotransferase, which confers resistance to hygromycin (Marsh (1984), Gene 32:481-485; see also Waldron et al. (1985), Plant Mol. Biol., 5:103-108; Zhijian et al. (1995), Plant Science 108:219-227; trpB, which allows cells to utilize indole in place of tryptophan; hisD, which allows cells to utilize histinol in place of histidine (Hartman (1988), Proc. Natl. Acad. Sci., USA 85:8047); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO (McConlogue (1987) in Current Communications in Molecular Biology, Cold Spring Harbor Laboratory); and deaminase from Aspergillus terreus, which confers resistance to Blasticidin S (Tamura (1995), Biosci. Biotechnol. Biochem. 59:2336-2338).

Additional selectable markers include, for example, a mutant acetolactate synthase, which confers imidazolinone or sulfonylurea resistance (Lee et al. (1988), EMBO J., 7:1241-1248), a mutant psbA, which confers resistance to atrazine (Smeda et al. (1993), Plant Physiol., 103:911-917, 1993), or a mutant protoporphyrinogen oxidase (see U.S. Pat. No. 5, 767, 373), or other markers conferring resistance to an herbicide such as glufosinate. Examples of suitable selectable marker genes include, but are not limited to: genes encoding resistance to chloramphenicol (Herrera Estrella et al. (1983), EMBO J., 2:987-992,); streptomycin (Jones et al. (1987) Mol. Gen. Genet., 210:86-91); spectinomycin (Bretagne-Sagnard et al. (1996) Transgenic Res., 5:131-137); bleomycin (Hille et al. (1990) Plant Mol. Biol., 7:171-176,); sulfonamide (Guerineau et al. (1990) Plant Mol. Biol., 15:127-136); bromoxynil (Stalker et al. (1988) Science, 242:419-423); glyphosate (Shaw et al. (1986), Science 233:478-481); phosphinothricin (DeBlock et al. (1987), EMBO J., 6:2513-2518), and the like.

One option for use of a selective gene is a glufosinate-resistance encoding DNA and in one embodiment can be the phosphinothricin acetyl transferase (pat), maize optimized pat gene or bar gene under the control of the Cassava Vein Mosaic Virus promoter. These genes confer resistance to bialaphos. See, (see, Wohlleben et al. (1988) Gene 70: 25-37); Gordon-Kamm et al. (1990), Plant Cell, 2:603; Uchimiya et al. (1993), BioTechnology 11:835, 1993; White et al. (1990) Nucl. Acids Res. 18:1062; Spencer et al. (1990), Theor. Appl. Genet. 79:625-631; and Anzai et al. (1989) Mol. Gen. Gen. 219:492). A version of the pat gene is the maize optimized pat gene, described in U.S. Pat. No. 6,096,947.

In addition, markers that facilitate identification of a plant cell containing the polynucleotide encoding the marker may be employed. Scorable or screenable markers are useful, where presence of the sequence produces a measurable product and can produce the product without destruction of the plant cell. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. (1987) EMBO J. 6(13): 3901-3907); and alkaline phosphatase. In a preferred embodiment, the marker used is beta-carotene or provitamin A (Ye et al. (2000) Science 287: 303-305). The gene has been used to enhance the nutrition of rice, but in this instance it is employed instead as a screenable marker, and the presence of the gene linked to a gene of interest is detected by the golden color provided. Unlike the situation where the gene is used for its nutritional contribution to the plant, a smaller amount of the protein suffices for marking purposes. Other screenable markers include the anthocyanin/flavonoid genes in general (See discussion at Taylor and Briggs (1990) Plant Cell, 2: 115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al. (1996) Plant Cell 8: 1171-1179; Scheffler et al. (1994) Mol. Gen. Genet. 242:40-48) and maize C2 (Wienand et al., Mol. Gen. Genet. (1986) 203:202-207); the B gene (Chandler et al. (1989) Plant Cell 1:1175-1183), the p1 gene (Grotewold et al. (1991) Proc. Natl. Acad. Sci USA, 88: 4587-4591; Grotewold et al., Cell (1994) 76: 543-553; Sidorenko et al. (1999) Plant Mol. Biol., 39: 11-19); the bronze locus genes (Ralston et al. (1988) Genetics 119:185-197; Nash et al. (1990) Plant Cell 2(11): 1039-1049), among others.

Further examples of suitable markers include the cyan fluorescent protein (CYP) gene (Bolte et al. (2004) J. Cell Science 117: 943-54 and Kato et al. (2002) Plant Physiol 129: 913-42), the yellow fluorescent protein gene (PHIYFP™ from Evrogen; see Bolte et al. (2004) J. Cell Science 117: 943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); a green fluorescent protein (GFP) gene (Sheen et al., Plant J. (1995) 8(5):777-84); and DsRed2 where plant cells transformed with the marker gene are red in color, and thus visually selectable (Dietrich et al. (2002) Biotechniques 2(2): 286-293). Additional examples include a β-lactamase gene (Sutcliffe (1978) Proc. Nat'l. Acad. Sci. U.S.A. 75:3737), which encodes an enzyme for which various chromogenic substrates are known (e.g., PADAC, a chromogenic cephalosporin); a xy1E gene (Zukowsky et al. (1983) Proc. Nat'l. Acad. Sci. U.S.A. 80:1101), which encodes a catechol dioxygenase that can convert chromogenic catechols; an α-amylase gene (Ikuta et al. (1990), Biotech. 8:241); and a tyrosinase gene (Katz et al. (1983) J. Gen. Microbiol. 129:2703), which encodes an enzyme capable of oxidizing tyrosine to DOPA and dopaquinone, which in turn condenses to form the easily detectable compound melanin. Clearly, many such markers are available and known to one skilled in the art.

In certain embodiments, the nucleotide sequence can be optionally combined with another nucleotide sequence of interest. The term “nucleotide sequence of interest” refers to a nucleic acid molecule (which may also be referred to as a polynucleotide) which can be a transcribed RNA molecule as well as DNA molecule, that encodes for a desired polypeptide or protein, but also may refer to nucleic acid molecules that do not constitute an entire gene, and which do not necessarily encode a polypeptide or protein (e.g., a promoter). For example, in certain embodiments the nucleic acid molecule can be combined or “stacked” with another that provides additional resistance or tolerance to glyphosate or another herbicide, and/or provides resistance to select insects or diseases and/or nutritional enhancements, and/or improved agronomic characteristics, and/or proteins or other products useful in feed, food, industrial, pharmaceutical or other uses. The “stacking” of two or more nucleic acid sequences of interest within a plant genome can be accomplished, for example, via conventional plant breeding using two or more events, transformation of a plant with a construct which contains the sequences of interest, re-transformation of a transgenic plant, or addition of new traits through targeted integration via homologous recombination.

Such nucleotide sequences of interest include, but are not limited to, those examples provided below:

1. Genes or Coding Sequence (e.g. iRNA) That Confer Resistance to Pests or Disease

(A) Plant Disease Resistance Genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. Examples of such genes include, the tomato Cf-9 gene for resistance to Cladosporium falvum (Jones et al., 1994 Science 266:789), tomato Pto gene, which encodes a protein kinase, for resistance to Pseudomonas syringae pv. tomato (Martin et al., 1993 Science 262:1432), and Arabidopsis RSSP2 gene for resistance to Pseudomonas syringae (Mindrinos et al., 1994 Cell 78:1089).

(B) A Bacillus thuringiensis protein, a derivative thereof or a synthetic polypeptide modeled thereon, such as, a nucleotide sequence of a Bt δ-endotoxin gene (Geiser et al. (1986) Gene 48:109), and a vegetative insecticidal (VIP) gene (see, e.g., Estruch et al. (1996) Proc. Natl. Acad. Sci. 93:5389-94). Moreover, DNA molecules encoding δ-endotoxin genes can be purchased from American Type Culture Collection (Rockville, Md.), under ATCC accession numbers 40098, 67136, 31995 and 31998.

(C) A lectin, such as, nucleotide sequences of several Clivia miniata mannose-binding lectin genes (Van Damme et al. (1994) Plant Molec. Biol. 24:825).

(D) A vitamin binding protein, such as avidin and avidin homologs which are useful as larvicides against insect pests. See U.S. Pat. No. 5,659,026.

(E) An enzyme inhibitor, e.g., a protease inhibitor or an amylase inhibitor. Examples of such genes include a rice cysteine proteinase inhibitor (Abe et al. (1987) J. Biol. Chem. 262:16793), a tobacco proteinase inhibitor I (Huub et al. (1993) Plant Molec. Biol. 21:985), and an α-amylase inhibitor (Sumitani et al. (1993) Biosci. Biotech. Biochem. 57:1243).

(F) An insect-specific hormone or pheromone such as an ecdysteroid and juvenile hormone a variant thereof, a mimetic based thereon, or an antagonist or agonist thereof, such as baculovirus expression of cloned juvenile hormone esterase, an inactivator of juvenile hormone (Hammock et al. (1990) Nature 344:458).

(G) An insect-specific peptide or neuropeptide which, upon expression, disrupts the physiology of the affected pest (J. Biol. Chem. 269:9). Examples of such genes include an insect diuretic hormone receptor (Regan, 1994), an allostatin identified in Diploptera punctata (Pratt, 1989), and insect-specific, paralytic neurotoxins (U.S. Pat. No. 5,266,361).

(H) An insect-specific venom produced in nature by a snake, a wasp, etc., such as a scorpion insectotoxic peptide (Pang (1992) Gene 116:165).

(I) An enzyme responsible for a hyperaccumulation of monoterpene, a sesquiterpene, a steroid, hydroxamic acid, a phenylpropanoid derivative or another non-protein molecule with insecticidal activity.

(J) An enzyme involved in the modification, including the post-translational modification, of a biologically active molecule; for example, glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. Examples of such genes include, a callas gene (PCT published application WO93/02197), chitinase-encoding sequences (which can be obtained, for example, from the ATCC under accession numbers 3999637 and 67152), tobacco hookworm chitinase (Kramer et al. (1993) Insect Molec. Biol. 23:691), and parsley ubi4-2 polyubiquitin gene (Kawalleck et al. (1993) Plant Molec. Biol. 21:673).

(K) A molecule that stimulates signal transduction. Examples of such molecules include nucleotide sequences for mung bean calmodulin cDNA clones (Botella et al. (1994) Plant Molec. Biol. 24:757) and a nucleotide sequence of a maize calmodulin cDNA clone (Griess et al. (1994) Plant Physiol. 104:1467).

(L) A hydrophobic moment peptide. See U.S. Pat. Nos. 5,659,026 and 5,607,914; the latter teaches synthetic antimicrobial peptides that confer disease resistance.

(M) A membrane permease, a channel former or a channel blocker, such as a cecropin-β lytic peptide analog (Jaynes et al. (1993) Plant Sci. 89:43) which renders transgenic tobacco plants resistant to Pseudomonas solanacearum.

(N) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. See, for example, Beachy et al. (1990) Ann. Rev. Phytopathol. 28:451.

(O) An insect-specific antibody or an immunotoxin derived therefrom. Thus, an antibody targeted to a critical metabolic function in the insect gut would inactivate an affected enzyme, killing the insect. For example, Taylor et al. (1994) Abstract #497, Seventh Int'l. Symposium on Molecular Plant-Microbe Interactions shows enzymatic inactivation in transgenic tobacco via production of single-chain antibody fragments.

(P) A virus-specific antibody. See, for example, Tavladoraki et al. (1993) Nature 266:469, which shows that transgenic plants expressing recombinant antibody genes are protected from virus attack.

(Q) A developmental-arrestive protein produced in nature by a pathogen or a parasite. Thus, fungal endo α-1,4-D polygalacturonases facilitate fungal colonization and plant nutrient release by solubilizing plant cell wall homo-α-1,4-D-galacturonase (Lamb et al. (1992) Bio/Technology 10: 1436). The cloning and characterization of a gene which encodes a bean endopolygalacturonase-inhibiting protein is described by Toubart et al. (1992) Plant J. 2:367.

(R) A developmental-arrestive protein produced in nature by a plant, such as the barley ribosome-inactivating gene that provides an increased resistance to fungal disease Longemann et al. (1992). Bio/Technology 10:3305.

(S) RNA interference, in which an RNA molecule is used to inhibit expression of a target gene. An RNA molecule in one example is partially or fully double stranded, which triggers a silencing response, resulting in cleavage of dsRNA into small interfering RNAs, which are then incorporated into a targeting complex that destroys homologous mRNAs. See, e.g., U.S. Pat. Nos. 6,506,559 and 6,573,099.

2. Genes That Confer Resistance to a Herbicide

(A) Genes encoding resistance or tolerance to a herbicide that inhibits the growing point or meristem, such as an imidazalinone, sulfonanilide or sulfonylurea herbicide. Exemplary genes in this category code for mutant acetolactate synthase (ALS) (Lee et al. (1988) EMBO J. 7:1241) also known as acetohydroxyacid synthase (AHAS) enzyme (Miki et al. (1990) Theor. Appl. Genet. 80:449).

(B) One or more additional genes encoding resistance or tolerance to glyphosate imparted by mutant EPSP synthase and aroA genes, or through metabolic inactivation by genes such as GAT (glyphosate acetyltransferase) or GOX (glyphosate oxidase) and other phosphono compounds such as glufosinate (pat and bar genes; DSM-2), and aryloxyphenoxypropionic acids and cyclohexanediones (ACCase inhibitor encoding genes). See, for example, U.S. Pat. No. 4,940,835, which discloses the nucleotide sequence of a form of EPSP which can confer glyphosate resistance. A DNA molecule encoding a mutant aroA gene can be obtained under ATCC Accession Number 39256, and the nucleotide sequence of the mutant gene is disclosed in U.S. Pat. No. 4,769,061. European patent application No. 0 333 033 and U.S. Pat. No. 4,975,374 disclose nucleotide sequences of glutamine synthetase genes which confer resistance to herbicides such as L-phosphinothricin. The nucleotide sequence of a phosphinothricinacetyl-transferase gene is provided in European application No. 242 246. De Greef et al. (1989) Bio/Technology 7:61 describes the production of transgenic plants that express chimeric bar genes coding for phosphinothricin acetyl transferase activity. Exemplary of genes conferring resistance to aryloxyphenoxypropionic acids and cyclohexanediones, such as sethoxydim and haloxyfop, are the Accl-S1, Accl-S2 and Accl-S3 genes described by Marshall et al. (1992) Theor. Appl. Genet. 83:435.

(C) Genes encoding resistance or tolerance to a herbicide that inhibits photosynthesis, such as a triazine (psbA and gs+genes) and a benzonitrile (nitrilase gene). Przibilla et al. (1991) Plant Cell 3:169 describes the use of plasmids encoding mutant psbA genes to transform Chlamydomonas. Nucleotide sequences for nitrilase genes are disclosed in U.S. Pat. No. 4,810,648, and DNA molecules containing these genes are available under ATCC accession numbers 53435, 67441 and 67442. Cloning and expression of DNA coding for a glutathione S-transferase is described by Hayes et al. (1992) Biochem. J. 285:173.

(D) Genes encoding resistance or tolerance to a herbicide that bind to hydroxyphenylpyruvate dioxygenases (HPPD), enzymes which catalyze the reaction in which para-hydroxyphenylpyruvate (HPP) is transformed into homogentisate. This includes herbicides such as isoxazoles (EP418175, EP470856, EP487352, EP527036, EP560482, EP682659, U.S. Pat. No. 5,424,276), in particular isoxaflutole, which is a selective herbicide for maize, diketonitriles (EP496630, EP496631), in particular 2-cyano-3-cyclopropyl-1-(2-SO2CH3-4-CF3 phenyl)propane- 1,3 -dione and 2-cyano-3 -cyclopropyl-1-(2-SO2CH3 -4-2,3Cl2phenyl)propane-1,3-dione, triketones (EP625505, EP625508, U.S. Pat. No. 5,506,195), in particular sulcotrione, and pyrazolinates. A gene that produces an overabundance of HPPD in plants can provide tolerance or resistance to such herbicides, including, for example, genes described in U.S. Pat. Nos. 6,268,549 and 6,245,968 and U.S. Patent Application, Publication No. 20030066102.

(E) Genes encoding resistance or tolerance to phenoxy auxin herbicides, such as 2,4-dichlorophenoxyacetic acid (2,4-D) and which may also confer resistance or tolerance to aryloxyphenoxypropionate (AOPP) herbicides. Examples of such genes include the α-ketoglutarate-dependent dioxygenase enzyme (aad-1) gene, described in U.S. Pat. No. 7,838,733.

(F) Genes encoding resistance or tolerance to phenoxy auxin herbicides, such as 2,4-dichlorophenoxyacetic acid (2,4-D) and which may also confer resistance or tolerance to pyridyloxy auxin herbicides, such as fluroxypyr or triclopyr. Examples of such genes include the α-ketoglutarate-dependent dioxygenase enzyme gene (aad-12), described in WO 2007/053482 A2.

(G) Genes encoding resistance or tolerance to dicamba (see, e.g., U.S. Patent Publication No. 20030135879).

(H) Genes providing resistance or tolerance to herbicides that inhibit protoporphyrinogen oxidase (PPO) (see U.S. Pat. No. 5,767,373).

(I) Genes providing resistance or tolerance to triazine herbicides (such as atrazine) and urea derivatives (such as diuron) herbicides which bind to core proteins of photosystem II reaction centers (PS II) (See Brussian et al., (1989) EMBO J. 8(4): 1237-1245.

3. Genes That Confer or Contribute to a Value-Added Trait

(A) Modified fatty acid metabolism, for example, by transforming maize or Brassica with an antisense gene or stearoyl-ACP desaturase to increase stearic acid content of the plant (Knultzon et al. (1992) Proc. Nat. Acad. Sci. USA 89:2624.

(B) Decreased phytate content

(1) Introduction of a phytase-encoding gene, such as the Aspergillus niger phytase gene (Van Hartingsveldt et al. (1993) Gene 127:87), enhances breakdown of phytate, adding more free phosphate to the transformed plant.

(2) A gene could be introduced that reduces phytate content. In maize, this, for example, could be accomplished by cloning and then reintroducing DNA associated with the single allele which is responsible for maize mutants characterized by low levels of phytic acid (Raboy et al. (1990) Maydica 35:383).

(C) Modified carbohydrate composition effected, for example, by transforming plants with a gene coding for an enzyme that alters the branching pattern of starch. Examples of such enzymes include, Streptococcus mucus fructosyltransferase gene (Shiroza et al. (1988) J. Bacteriol. 170:810), Bacillus subtilis levansucrase gene (Steinmetz et al. (1985) Mol. Gen. Genet. 200: 220), Bacillus licheniformis α-amylase (Pen et al. (1992) Bio/Technology 10: 292), tomato invertase genes (Elliot et al., 1993), barley amylase gene (Sogaard et al. (1993) J. Biol. Chem. 268:22480), and maize endosperm starch branching enzyme II (Fisher et al. (1993) Plant Physiol. 102:10450).

The sequence of interest can also be a nucleotide sequence introduced into a predetermined area of the plant genome through homologous recombination. Methods to stably integrate a polynucleotide sequence within a specific chromosomal site of a plant cell via homologous recombination have been described within the art. For instance, site specific integration as described in US Patent Application Publication No. 2009/0111188 A1 involves the use of recombinases or integrases to mediate the introduction of a donor polynucleotide sequence into a chromosomal target. In addition, International Patent Application No. WO 2008/021207 describes zinc finger mediated-homologous recombination to stably integrate one or more donor polynucleotide sequences within specific locations of the genome. The use of recombinases such as FLP/FRT as described in U.S. Pat. No. 6,720,475, or CRE/LOX as described in U.S. Pat. No. 5,658,772, can be utilized to stably integrate a polynucleotide sequence into a specific chromosomal site. Finally, the use of meganucleases for targeting donor polynucleotides into a specific chromosomal location was described in Puchta et al. (1996) Proc. Nat'l Acad. Sci. USA 93: 5055-5060.

Other various methods for site specific integration within plant cells are generally known and applicable (Kumar et al. (2001), Trends in Plant Sci. 6(4): 155-159). Furthermore, site-specific recombination systems that have been identified in several prokaryotic and lower eukaryotic organisms may be applied for use in plants. Examples of such systems include, but are not limited too; the R/RS recombinase system from the pSR1 plasmid of the yeast Zygosaccharomyces rouxii (Araki et al. (1985) J. Mol. Biol. 182: 191-203), and the Gin/gix system of phage Mu (Maeser and Kahlmann (1991) Mol. Gen. Genet. 230: 170-176).

While certain example Agrobacterium strains are described herein, the functionality of the novel transformation methods discussed could be moved to other Agrobacterium strains with the same criteria. Examples of other strains that could be used with the novel transformation method described herein include, but are not limited to, Agrobacterium tumefaciens strain C58, Agrobacterium tumefaciens strain Chry5, Agrobacterium rhizogenes strains, Agrobacterium tumefaciens strain EHA101, Agrobacterium tumefaciens strain EHA105, Agrobacterium tumefaciens strain MOG101, and Agrobacterium tumefaciens strain T37. Modified versions of such strains are described with more particularity in International Patent Application No. WO 2012/106222 A2, incorporated herein by reference.

In embodiments, mature soybean seeds are sterilized prior to infection. Seeds may be sterilized using chlorine gas, mercuric chloride, immersion in sodium hypochloride, immersion in sodium carbonate, or other suitable methods known in the art. In embodiments, the seeds are imbibed using sterile water or other suitable hypotonic solutions. Imbibing the seeds for 6-24 hours softens the seeds, saturates the cotyledons, and improves later shoot induction. Longer periods of imbibing may also be used, for example, up to 48 hours.

The split-seed soybean is prepared by splitting the cotyledons of the seeds along the hilum to separate the cotyledons, then removing the seed coat. Removal of a portion of the embryo axis leaves part of the axis attached to the cotyledons prior to transformation. Typically, between ⅓ and ½ of the embryo axis is left attached at the nodal end of the cotyledon.

Wounding of the split soybean seed is not required with the disclosed method, but is reported to increase transformation efficiency using other methods, including the cotyledonary node method and the meristem explant method. Wounding of the plant material may be facilitated by cutting, abrading, piercing, sonication, plasma wounding, or vacuum infiltration. Split soybean seeds comprising a portion of an embryonic axis are typically inoculated with Agrobacterium culture containing a suitable genetic construct for about 0.5 to 3.0 hours, more typically for about 0.5 hours, followed by a period of co-cultivation on suitable medium for up to about 5 days. Explants which putatively contain a copy of the transgene arise from the culturing of the transformed split soybean seeds comprising a portion of an embryonic axis. These explants are identified and isolated for further tissue propagation.

Shoot induction may be facilitated by culturing explants in suitable induction media for a period of approximately two weeks, followed by culturing in media containing a selectable agent, such as glufosinate, for another two weeks. Alternating between media without a selectable agent, and media with a selectable agent, is preferred, but other protocols wherein the media always comprises a selectable agent may be successfully employed. Following a period of shoot induction, a tissue isolate containing a portion of the embryonic axis may be excised, and transferred to a suitable shoot elongation medium. In embodiments, the cotyledons may be removed, and a flush shoot pad excised containing the embryonic axis may be excised, by cutting at the base of the cotyledon. See Example 2.

Typically, one or more selective agents are applied to the split-seed explants following transformation. The selective agent kills or retards the grown of non-transformed soybean cells, and may help to eliminate the residual Agrobacterium cells. Suitable agents include glufosinate or Bialaphos. Other suitable agents include, but are not limited to, the herbicide glyphosate or the herbicide 2,4-D which acts as both a selectable agent and shoot-inducing hormone. In addition, the selective agents can include various antibiotics, including spectinomycin, kanamycin, neomycin, paromomycin, gentamicin, and G418, depending on the selectable marker used. Depending on the agent used, selection for one to seven days may be appropriate.

Rooting of elongated shoots may be encouraged using suitable agents, including, but not limited to varying concentrations of auxins and cytokinins. For example the auxin, indole 3-butryic acid (IBA), may be incorporated into cell tissue culture medium that is used prior to transfer of the plant material to suitable rooting media known to those of skill in the art. Root formation takes approximately 1-4 weeks, more typically 1-2 weeks after exposure to IBA.

Cultivation of growing shoots may be accomplished by methods generally known in the art, leading to mature transgenic soybean plants. See, e.g., Example 2.

The presence of successful transgenic events may be confirmed using techniques known in the art, including, but not limited to TAQMAN™, PCR analysis, and Southern analysis of integrated selectable markers and/or reporter gene constructs in the soybean at any stage after infection and co-cultivation with Agrobacterium; phenotypic assay for plants or plant germplasm displaying evidence of a reporter construct; or selection of explants on suitable selection media.

In embodiments, the disclosed method may be used to facilitate breeding programs for the development of inbred soybean lines expressing genes of interest, and the development of elite soybean cultivars. Inbred soybean lines comprising stably-integrated transgenes may be crossed with other inbred soybean lines to produce hybrid plants expressing genes of interest. Introgression of a desired trait into elite soybean lines and hybrids may be rapidly achieved using the disclosed method and methods known in the art.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

The following Examples are provided to illustrate particular features and/or aspects of the disclosure. These Examples should not be construed to limit the disclosure to the particular features or aspects described therein.

### EXAMPLE 1

The following example demonstrates the identification and cold treatment of pods having immature embryos in accordance with the invention. For production of immature zygotic embryos, seeds of soybean [Glycine max (L.) Merrill] cultivars Maverick and Jack were grown in a greenhouse at ambient temperatures, with illumination from high-pressure sodium lamps to maintain ideal flowering conditions with a 14-hour photoperiod. Five plants were grown in 10 inch pots containing a 2:2:1 mixture of soil (Maury silt loam): Promix™ (Premier Brands, New Rochelle, N.Y.): sand. The plants were fertilized weekly with Peter's 20-20-20™ fertilizer (The Scotts Company, Marysville Ohio).

Five weeks after planting, or from about 7 to 14 days after flowering, pods larger than 0.9 cm in width were harvested. Harvested pods were screened for the presence of immature embryos under a trans-illuminated stereoscope. Pods having 2-3 embryos were selected and refrigerated at 4° C. for 7-9 days. Cold-treated pods were then surface sterilized by washing for 30 seconds with 70% EtOH, then with 10% bleach containing TWEEN-20(Sigma-Aldrich, St. Louis Mo.) for 10 minutes with gentle agitation. Bleach was decanted and sterilized pods were rinsed 3 times with sterile water for 5 minutes with gentle agitation.

### EXAMPLE 2

The following example demonstrates susceptibility of whole immature embryos and split embryos to form callus on 2, 4-D rich media.

Pods were cold treated and sterilized as described in Example 1. Pods were subsequently screened using backlighting on a trans-illuminated stereoscope to determine the position of immature embryos in each pod. For each pod selected, two cuts were then made on each end and then one long cut was made longitudinally along the curved part of the pod. While making the long cut, enough pod tissue was cut away to expose the interior of the pod cavity. Next, the pod was disassembled by grasping the interior pod cavity and detaching the embryos from the pod. Slight pressure was applied on each embryo when removing it from the pod. Immature embryos of 2 mm to 9 mm in length were selected for use.

Whole immature embryos were placed on 2, 4-D rich semi-solid media (referred to as “SE-40” herein) containing MS basal salts 4.33 g/L (Murashige and Skoog, Physiol. Plant (1962) 15(3): 473-497), Gamborg's B5 vitamins 1 ml/L (Gamborg et al., Exp. Cell Res. (1968), 50: 151-158), sucrose 30 g/L, 2,4-D 40 mg/L, GEL-RITE 2g/L (Sigma-Aldrich). Callus initiation was observed approximately three weeks after placement on SE-40 media. Generally, smaller immature embryos were found to be better callus producers. For Jack and Maverick varieties, the majority of callus producers were immature embryos of less than 5 mm in length. Furthermore, callus-producing whole embryos in this example did not generate somatic embryo explant. Thus, callus-producing whole immature embryos differ from the split embryos described in the following example.

### EXAMPLE 3

This example provides a demonstration of the process for longitudinally bisecting immature embryos to form split embryos and the use of split embryos to generate embryogenic callus and somatic embryos in accordance with the invention. The following also demonstrates an example of the effect of explant size on the formation of callus and somatic embryonic tissue.

Pods were cold treated and sterilized and immature embryos isolated from the pods as described in Examples 1 and 2. Selected immature embryos of 3 to 9 mm were cut longitudinally along the hilum to bisect the two cotyledons with intact embryonic axis. Each of these split immature embryo explants, with its intact embryonic axis, was placed on 2, 4-D rich semi-solid media (SE-40) such that its abaxial side was in contact with the semi-solid media and its adaxial (flat) side was oriented upwards, away from the semi-solid media surface. After three to four weeks on SE-40 media, split immature embryos were assayed for their ability to induce embryogenic callus. Table 1 shows the following information for the split immature embryo explants: cultivar name, immature embryo size range, total number of split immature embryo explants that were cultured on SE-40 media, and frequency of split immature embryo explants that formed embryogenic callus, which frequency is shown as the number of explants that induced embryogenic callus and the percentage equal to ([number of embryos that induced callus]/[total number of explants cultured on CI media]*100%). The results in Table 1 demonstrate that callus induction frequency increased with larger split immature embryos to a maximum at greater than 5 mm to 6 mm (97%) in Jack and at greater than 6 mm (99%) in Maverick.

TABLE 1

| | | | Total Number | |
|---|---|---|---|---|
| | | | of Explants | Number of Explants that |
| | | | Cultured on | Induced Embryogenic Callus |
| Cultivar | Embryo Size | | SE-40 Media | (% Total) |
| Jack | 2 to 3 | mm | 2 | 1 (50%) |
| Jack | 3 to 4 | mm | 60 | 22 (37%) |
| Jack | 4 to 5 | mm | 50 | 39 (78%) |
| Jack | 5 to 6 | mm | 31 | 30 (97%) |
| Jack | 6+ | mm | 31 | 30 (97%) |
| Maverick | 2 to 3 | mm | 9 | 2 (22%) |
| Maverick | 3 to 4 | mm | 83 | 47 (57%) |
| Maverick | 4 to 5 | mm | 84 | 68 (81%) |
| Maverick | 5 to 6 | mm | 65 | 61 (94%) |
| Maverick | 6+ | mm | 97 | 96 (99%) |

After four weeks on SE-40 media, split embryos were transferred to somatic embryo germination media (SEGM) without 2,4-D. At sixteen days of culturing on callus induction media, callused immature embryo explants were imaged using a Leica M165 FC™ stereo scope (Leica Microsystems, Heerbrugg, Switzerland). From the collected images, each transferred immature embryo was assigned a positive or negative score for the presence of somatic embryo to investigate the correlation between immature embryo size and susceptibility to form somatic embryo tissue. Table 2 shows the size ranges of immature embryos, and for each size range the total number of immature embryos that were cultured SEGM media for both Jack and Maverick varieties as well as the frequency of somatic embryo (SE) formed for both Jack and Maverick varieties, wherein the frequency is shown as the number of SE formed and as the percentage equal to ([number of SE formed]/[total number of immature embryos cultured on SEGM media]*100%). The results in Table 2 demonstrated a correlation between immature embryo size and SE formation. The highest frequency of somatic embryo induction was observed with 4 mm to 5 mm sized embryo explants in Jack (73%), while 5 mm to 6 mm sized embryos produced the highest frequency of somatic embryos in Maverick (69%).

TABLE 2

| | | Immature Jack | Number | Immature | Number |
|---|---|---|---|---|---|
| Size of | | Embryos | of SE | Maverick | of SE |
| Immature | | on SEGM | Formed | Embryos on | Formed (%) |
| Embryos | | Media | (%) Jack | SEGM Media | Maverick |
| 2 to 3 | mm | 1 | 0 (0%) | 2 | 0 (0%) |
| 3 to 4 | mm | 22 | 10 (45%) | 47 | 17 (36%) |
| 4 to 5 | mm | 39 | 30 (77%) | 68 | 37 (54%) |
| 5 to 6 | mm | 30 | 22 (73%) | 61 | 42 (69%) |
| 6+ | mm | 30 | 21 (70%) | 96 | 53 (55%) |

### EXAMPLE 4

The following example demonstrates the use of cold treatment on immature embryos in soybean pods in accordance with the invention to increase both production of embryogenic callus and somatic embryo formation. This example also demonstrates the effects of cold treatment for different lengths of time and that tissues induced with cold treatment are fully capable of regeneration.

The number of days of cold treatment (4 ° C.) was varied to determine its effect on embryogenic callus induction and somatic embryo (SE) formation and results are shown in Table 3. The results in Table 3 demonstrate that storage or cold treatment of immature embryos for 8 days increased the production and development of both callus and SE tissue. In this example, more than 8 days of cold treatment decreased the production and development of both callus and SE tissue. The highest frequency of callus production (92%) was observed after an 8-day period of cold treatment, as compared to no cold treatment (71%). Additional days of cold treatment (e.g., 9 days or greater) reduced the proliferation of callus tissues. Lowest frequency (57%) of callus was produced by an 11-day cold treatment period. Similarly, somatic embryo (SE) induction (70%) was highest using 8-day cold treatment period, as compared to no cold treatment (21%). Longer periods of cold treatment generally decreased SE induction. The lowest frequency of SE induction (31%) was produced by an 11-day cold treatment period. However, even this 11-day cold treatment period frequency was higher than the frequency of SE induction (21%) with no cold treatment. Across the different genotypes tested, increased frequency of callus and SE production as a result of cold treatment of immature embryos was statistically significant, especially SE production at 8 days of cold treatment.

TABLE 3

| | | No. of Immature | No. of Immature |
|---|---|---|---|
| No. of Days | Total No. of | Embryos Explants | Embryo Explants |
| of Cold | Immature Embryo | with Embryogenic | with Somatic |
| Treatment | Explants Cultured | Callus (%) | Embryos (%) |
| 0 | 68 | 48 (71%) | 14 (21%) |
| 2 | 78 | 66 (85%) | 41 (53%) |
| 5 | 16 | 14 (88%) | 7 (44%) |
| 7 | 126 | 112 (89%) | 74 (59%) |
| 8 | 50 | 46 (92%) | 35 (70%) |
| 10 | 27 | 26 (96%) | 16 (59%) |
| 11 | 147 | 84 (57%) | 45 (31%) |

Maverick did show better induction of callus when compared to Jack. However, the somatic embryo tissue production was found to be genotype independent. Tissues induced with cold treatment were found to be fully capable of regeneration.

### EXAMPLE 5

The following example demonstrates the use of plasmolysis treatment in accordance with the invention to increase somatic embryo formation.

Before being transferred to 2,4-D rich semi-solid media (SE-40), split immature embryo explants were cultured for 0, 4, or 24 hours on plasmolysis media (MS Basal Medium with vitamins 4.4 g/L, Mannitol 0.4M, Sorbitol 0.4M, Gelrite 2.3 g/L, Magnesium Chloride Hexahydrate 0.5mM). The results shown in Table 4 demonstrate that plasmolysis treatment improved the percentage of somatic embryo (SE) regeneration. In this example, 4-hour culture on plasmolysis media was optimal for Jack soybean explants and resulted in 86% somatic embryo development. For Maverick soybean explants, 24-hour culture on plasmolysis media was optimal and resulted in 67% somatic embryo development.

TABLE 4

| | | | Number of | |
|---|---|---|---|---|
| | | Number | Immature Embryos | % Somatic |
| | Plasmolysis | Immature | that Produced | Embryo |
| Genotype | Treatment | Embryos | Somatic Embryos | Regeneration |
| Jack | 0 Hour | 20 | 11 | 55% |
| | 4 Hour | 50 | 43 | 86% |
| | 24 Hour | 30 | 19 | 63% |
| Maverick | 0 Hour | 20 | 1 | 5% |
| | 4 Hour | 50 | 11 | 22% |
| | 24 Hour | 30 | 20 | 67% |

### EXAMPLE 6

The following example demonstrates Agrobacterium-mediated transformation of split immature embryo explants prepared using cold treatment in accordance with the invention. This example also demonstrates the regeneration of stably transformed plants from transformed explants.

Single binary vector designated pDAB9381 was constructed to contain two plant transcription units (PTUs). The first PTU contains Arabidopsis thaliana ubiquitin-10 promoter (AtUbi10 promoter) operably linked to yellow fluorescence protein YFP coding sequence (PhiYFP). The YFP coding sequence contains an intron isolated from the Solanum tuberosum, light specific tissue inducible LS-1 gene (ST-LS1 intron; Genbank Accession No. X04753) and is terminated by the Agrobacterium tumefaciens open reading frame-23 3′ untranslated region (AtuORF23 3′UTR). The second PTU contains the isopentenyltransferase coding sequence (IPT CDS) (Genbank Accession No. X00639.1) and also contains the Cassava Vein Mosaic Virus promoter (CsVMV promoter operably linked to an herbicide-resistance selectable marker coding sequence, terminated by the A. tumefaciens open reading frame-13′ untranslated region (AtuORF1 3′UTR). This binary vector pDAB9381 was mobilized into the Agrobacterium tumefaciens strains of EHA105 using electroporation. Individual colonies were identified which grew up on YEP media containing the antibiotic spectinomycin. Single colonies were isolated and the presence of the pDAB9381 binary vector was confirmed via restriction enzyme digestion.

Cold-treated, split immature embryos with an intact embryogenic axis were prepared as described above and then transformed with pDAB9381 via the Agrobacterium-mediated plant method of US Patent Application Publication 2014/0173774 A1 (Pareddy et al). More particularly, split immature embryo explants of Maverick and Jack varieties were cold-treated, placed in Agrobacterium strain EHA105 transformation solution containing binary vector for 30 minutes, and then explants were placed on co-cultivation media containing herbicide selection agent for 5 days. Most of the smaller embryos turned white after this selection. Larger embryos did not turn white. Agrobacterium-treated immature embryos were subsequently placed on S-1 media containing selection agents for 4 weeks to generate callus. The resulting callus was screened for YFP expression via microscopy and the YFP-expressing callus tissues were identified and determined to contain transiently expressing copies of the YFP-encoding transgene. Table 5 shows the number of explants that were placed on co-cultivation (CC) media and the resulting number and percent frequency of explants that formed somatic embryo (SE) tissue expressing YFP on S-1 media, wherein the percent frequency is equal to ([number of SE Expressing YFP on S-1 Media]/[number of explants on CC media]*100%]).

TABLE 5

| | Number of | |
|---|---|---|
| | Explants on CC | Number of SE Expressing YFP on S-1 |
| Genotype | Media | Media (% Frequency) |
| Jack | 39 | 28 (71.7%) |
| Maverick | 43 | 27 (62.7%) |

Stably transformed plant tissues are selected and cultured into whole plants on tissue culture media components. The resulting T0 whole plants are selfed and the T1 generations of plants are assayed using molecular confirmation to confirm transgenes integrated within the plant genome.

### EXAMPLE 7

The following example demonstrates biolistic (particle bombardment) mediated transformation of split immature embryo explants prepared using cold treatment in accordance with the invention. This example also demonstrates the regeneration of stably transformed transgenic plants in accordance with the invention.

Cold-treated, split immature embryos with intact embryogenic axis were prepared as described above in Examples 1 and 2. After plasmolysis treatment for four hours, explant tissue was transformed using biolistic-mediated transformation with construct pDAB113639. Biolistic transformation involved gene gun bombardment using 0.6 μm gold particles, 900 PSI rupture disks, and 6 cm target distance. The pDAB 113639 construct contains an RFP (red fluorescent protein) reporter gene and the HPT (hygromycin resistance transgene) selectable marker gene.

After bombardment, explants were placed on 2,4-D rich solid SE40 media (MS Basal Salts 4.33g L-1, Gamborg B5 Vitamins 1000× 1 ml L-1, 40 mg L-12,4-D, 3% Sucrose, 0.2% Gelrite, pH 7) (Bailey et al. (1993) Plant Science, 93: 117-120) for 1 week, then transferred to 2,4-D rich semi-solid media (SE-40+10 μg/ml hygromycin) for 2 weeks, 2,4-D rich semi-solid media (SE-40 +25 μg/ml hygromycin) for 1 week, and subsequently placed on somatic embryo germination and maturation (SEGM) media (MS Basal Salts 4.33g L-1, Gamborg B5 Vitamins 1000× 1 ml L-1, 3% Sucrose, 0.2% Gelrite) media (+25 μg/m1 hygromycin; Finer, 1999) for 4 weeks. Expression of the RFP protein was visualized via fluorescent microscopy to confirm transformation of regenerated plant tissues.

Having applied selection in the induction phase, immature embryos were transferred to SEGM media for 2 weeks, during which time, somatic embryos began to form. Two alternative methods of somatic embryo maturation were tested by (1) continuing culture on solid SEGM media or (2) transferring to liquid culture soybean histodifferentiation and Maturation (SHaM) media (MS Micronutrient 10× 100 ml L-1, 3% Sucrose, 3.5 mM Ammonium Sulfate, 2 mM Anhydrous Calcium Chloride, 1.4 mM Monobasic Potassium Phosphate, 10 mM Potassium Nitrate, 1.5 mM Anhydrous Magnesium Sulfate, 0.16M Sorbitol, Gamborg B5 Vitamins 1000× 1 ml L-1, 30 mM Glutamine, 1 mM Methionine) (Schmidt et al. (2005) Plant Cell Reports, 24: 383-391). Table 6 shows the results of particle bombardment of Jack and Maverick explants. In particular, Table 6 shows the following information for each genotype and somatic embryo maturation media tested: number of explants that were bombarded; number of explants that formed somatic embryo tissue (SE); percentage that formed SE which is equal to ([number of explants formed SE]/[number of explants bombarded]*100%), total number of SE produced (which indicates the productivity or embryogenic potential of the immature embryos treated); conversion percentage (which indicates how many somatic embryos were converted into plantlets and can vary based on plant variety), number of transgenic plants confirmed by quantitative polymerase chain reaction (qPCR), transformation frequency percentage which is equal to ([number of confirmed transgenic plants]/[number of explants bombarded]*100%).

TABLE 6

| | Media | | | |
|---|---|---|---|---|
| | SEGM | | SHaM | |
| | Genotype | | | |
| | Jack | Maverick | Jack | Maverick |
| Number Explants | 290 | 850 | 30 | 30 |
| Bombarded | | | | |
| Number of Explants that | 60 | 140 | 14 | 8 |
| Formed SE | | | | |
| SE Frequency Percentage | 21% | 16% | 47% | 27% |
| Total Number of SE | 223 | 294 | 178 | 19 |
| Produced | | | | |
| Conversion Percentage | 19% | 26% | 12% | 37% |
| Number Confirmed | 10 | 11 | 5 | 3 |
| Transgenic Plants | | | | |
| Transformation Frequency | 3.4% | 1.3% | 16.7% | 10.0% |
| Percentage | | | | |

Improvements in the induction phase provided by the disclosed methods of the inventions can be tracked by improved total number of SE produced and/or by improved number of somatic embryos per immature embryo (“productivity of the immature embryo”). As shown in Table 6, generally, a higher frequency of somatic embryo production was observed in SHaM liquid media for both cultivars and higher number of somatic embryos was produced per immature embryo explant that was bombarded.

Transformed plant tissues were cultured on tissue culture media to generate whole transgenic plants having the transgene (comprising the reporter marker and selectable marker of pDAB 113639) stably integrated into their genome. The foregoing results demonstrate that the improved somatic embryogenesis provided by the invention was directly linked to improved transformation frequency via an expanded pool of transformable somatic embryo tissue.

### EXAMPLE 8

The following example demonstrates the heritability of the stable transgenic events created as described in Example 7 and confirms that the transgenic events segregated appropriately in all soybean varieties that were tested. The T0 generation events of Example 7 that tested positive for the transgene were allowed to set seed. Seed was harvested, sowed, and then grown in the greenhouse to generate T1 generation plants. When T1 plants had grown to V3 vegetative stage, leaf samples from the seedlings were analyzed by qPCR. This analysis demonstrated the presence of both RFP and HPT transgene markers in the T1 plant genome. Transgenic events of Jack and Maverick were both found to be heritable and segregated as shown by the results in Table 7.

TABLE 7

| | Total T₁ | # of seedlings | # of seedlings |
|---|---|---|---|
| | seedlings | positive for | negative for |
| Genotype | tested | transgene | transgene |
| Jack | 13 | 4 | 9 |
| Maverick | 14 | 10 | 4 |

The data in Table 7 confirm that the methods of the invention produce a variety of heritable events in different soybean genotype backgrounds.

### EXAMPLE 9

The following example demonstrates the use of additional selectable markers in the direct transformation method using biolistics-mediated transformation of split immature embryo explants. PAT (phosphinothricin-N-acetyltransferase) and DSM2 (Dow selectable marker) are selectable marker genes that provide resistance to glufosinate ammonium (GLA). DGT28 (Dow glyphosate tolerance) is a selectable marker gene that provides resistance to glyphosate.

Cold-treated, split immature embryos with intact embryogenic axis were prepared as described above in Examples 1 and 2. After plasmolysis treatment for four hours, explant tissue was subject to biolistic transformation with constructs pDAB9381 or pDAB126244 or pDAB126276. The pDAB9381 construct contains the YFP reporter gene and the PAT selectable marker gene. The pDAB126244 construct contains the RFP reporter gene and the DGT28 selectable marker gene. The pDAB126276 construct contains the YFP (yellow fluorescent protein) reporter gene and the DSM2 selectable marker gene. Biolistic transformation involved gene gun bombardment using 0.6 μm gold particles, 900 PSI rupture disks, and 6 cm target distance.

After bombardment, explants were placed on 2,4-D rich solid SE40 media (MS Basal Salts 4.33g L-1, Gamborg B5 Vitamins 1000× 1 ml L-1, 40 mg L-1 2,4-D, 3% Sucrose, 0.2% Gelrite, pH 7) (Bailey et al. (1993)) for 1 week, then transferred to 2,4-D rich semi-solid media (SE-40) containing selection agent (1.5 mg/L Glufosinate Ammonium (“GLA”) or 0.25 mM glyphosate) for 3 weeks. Subsequently explants were placed on somatic embryo germination and maturation (SEGM) media (MS Basal Salts 4.33g L-1, Gamborg B5 Vitamins 1000× 1 ml L-1, 3% Sucrose, 0.2% Gelrite) media for 4 weeks. Expression of the YFP/RFP protein was visualized via fluorescent microscopy to confirm transformation of regenerated plant tissues.

Table 8 shows the following results for bombardment with each construct containing selectable marker PAT, DSM2 or DGT28: number of explants that were bombarded, number of transgenic somatic embryos produced and number of transgenic somatic embryos that converted into plantlets and were successfully established in the greenhouse. The percent number of transgenic somatic embryos produced is ([number of explants regenerating transgenic SE/number of explants bombarded]*100%). The percent number of transgenic plants established in the greenhouse is ([number of independent transgenic events established in the greenhouse/number of explants bombarded]*100%). Table 8 shows selectable marker used, the selection agent and application concentration, number of immature embryos (IE) bombarded, number transgenic somatic embryos (TSE) produced from bombarded IE along with corresponding “% TSE” which is ([TSE produced]/[IE bombarded]×100%), and the number of transgenic plants that were regenerated in the greenhouse from TSE along with corresponding transformation frequency percentage or “% TF” which is ([transgenic plants regenerated in greenhouse]/[IE bombarded]×100%).

TABLE 8

| | | | | Transgenic |
|---|---|---|---|---|
| Selectable | Selection agent | | Transgenic | Plants |
| Marker | (Application | Number of IE | SE | from TSE |
| Gene | Concentration) | Bombarded | (% TSE) | (% TF) |
| PAT | GLA | 450 | 17 (3.8%) | 5 (1.1%) |
| | (1.5 mg/L) | | | |
| DSM2 | GLA | 809 | 37 (4.7%) | 8 (1.0%) |
| | (1.5 mg/L) | | | |
| DGT28 | Glyphosate | 325 | 2 (0.6%) | 2 (0.6%) |
| | (0.25 mM) | | | |

As shown in Table 8, the methods of the invention were used to generate transgenic somatic embryos and plants with three different selectable marker genes that provide resistance to two different plant selection agents. Further, transgenic somatic embryos were regenerated into transgenic plants containing each selectable marker.

What may be claimed is:

1. A method for embryogenic callus production, wherein the method comprises:
selecting one or more immature soybean embryos;
subjecting one or more immature embryos to cold treatment; and
inducing the cold-treated embryos to form embryogenic callus.

2. The method of claim 1, wherein
the immature embryos are subjected to cold-treatment while in their pods or after being isolated from other pod tissues; and
the selected immature embryos have a length of 6 mm or greater.

3. The method of claim 1, wherein
the selected one or more immature embryos are isolated from their pods and
the one or more selected embryo explants are bisected to form split seed embryo explants.

4. The method of claim 1, wherein the immature embryos are harvested from a recalcitrant variety of Glycine max.

5. The method of claim 1, wherein the method comprises subjecting the one or more immature embryos to cold treatment for up to 8 days.

6. The method of claim 1, wherein the method further comprises subjecting the selected one or more immature embryos to plasmolysis treatment.

7. The method of any one of claim 6, wherein the immature embryos are subjected to plasmolysis treatment for 4-24 hours.

8. The method of claim 1, wherein the method further comprises inducing the formation of somatic embryo tissue.

9. The method of claim 1, wherein the method comprises inducing callus formation using growth media containing an auxin.

10. The method of claim 1, wherein the selected one or more immature embryos are translucent green.

11. The method of claim 1, wherein the method further includes
harvesting one or more soybean pods 7-14 days after flowering, wherein optionally the harvested pods containing the one or more selected immature embryos are larger than 9 mm in width; and
selecting the one or more selected immature embryos from the harvested pods.

12. A method for soybean transformation, wherein the method comprises:
selecting a plurality of immature soybean embryos;
subjecting the immature embryos to cold treatment;
transforming the cold-treated immature zygotic embryos with one or more exogenous transgenes; and
inducing the transformed zygotic embryos to form somatic embryos and thereby produce transgenic somatic embryo tissue.

13. A method for soybean transformation, wherein the method comprises:
selecting a plurality of immature soybean embryos;
subjecting the immature embryos to cold treatment;
inducing the cold-treated embryos to form somatic embryos; and
transforming the somatic embryos with one or more exogenous transgenes to thereby produce transgenic somatic embryo tissue.

14. The method of claim 13, wherein the method further comprises regenerating the transgenic somatic embryo tissue into a transgenic soybean plant comprising the one or more exogenous transgenes.

15. The method of claim 13, wherein the one or more selected immature embryos have a length of 6 mm or greater.

16. The method of claim 13, wherein
the one or more selected immature embryos are isolated from their pods and
the one or more selected embryo explants are bisected to form split seed embryo explants.

17. The method of claim 13, wherein, wherein the immature zygotic embryos are harvested from a recalcitrant variety of Glycine max.

18. The method of claim 13, wherein the method comprises subjecting the one or more immature embryos to one or more of the following additional steps: (i) cold treatment for up to 8 days, (ii) plasmolysis treatment, or (iii) plasmolysis treatment for 4-24 hours.

19. The method of claim 13, wherein the method comprises inducing callus formation using growth media containing an auxin.

20. The method of claim 13, wherein the selected one or more immature embryos are translucent green.

21. The method of claim 13, wherein the method further includes
harvesting one or more soybean pods 7-14 days after flowering, wherein optionally the harvested pods containing the one or more selected immature embryos are larger than 9 mm in width; and
selecting the one or more selected immature embryos from the harvested pods.

22. The method of claim 13, wherein
the transforming comprises using biolistic-mediated transformation; and
the one or more exogenous transgenes comprises a selectable marker gene.

23. The method of claim 22, wherein the selectable marker gene provides resistance to glufosinate, glyphosate, or hygromycin.

24. The method of claim 13, wherein the method further comprises maturing the transgenic somatic embryo tissue using soybean histodifferentiation and maturation (SHaM) media or somatic embryo germination and maturation (SEGM) media.
